(19) United States
(12) Patent Application Publication
(10) Pub. No.: US 2007/0243599 A1
(43) Pub. Date: Oct. 18, 2007

(54) METHOD FOR DETERMINING THE PHENOTYPE OF CELLS

(75) Inventor: Sicco Popma, Chalfont, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Centocor Research & Development, Inc., Radnor, PA (US)

(21) Appl. No.: 11/618,168

(22) Filed: Dec. 29, 2006

Related U.S. Application Data
(60) Provisional application No. 60/755,611, filed on Dec. 30, 2005.

(51) Int. Cl. C12M 1/34

(52) U.S. Cl. 435/287.1

(57) ABSTRACT

The present invention provides a system, a device, and a kit for comparing the phenotypic state of a first unknown sample material with a sample material of a known phenotypic state. The present invention also provides a method of using the system, device and kit.

### FIELD OF THE INVENTION

This invention is directed to methods for the rapid, efficient and accurate determination of the phenotype of cells. In particular, this invention is directed to methods employing antibodies.

### BACKGROUND

The surface of every cell in the body is coated with proteins. These cell surface proteins have several functions. For example, the cell surface proteins may be receptors, which have the capability of selectively binding or adhering to other “signaling” molecules. Alternatively, these proteins may be functional or structural. For example, the protein may facilitate the adherence of the cell to a substrate, or the protein may facilitate the secretion of a molecule. Each cell type, for example a liver cell, has a certain combination of proteins on their surface that makes them distinguishable from other kinds of cells or cell systems.

The expression of cell surface proteins on a given cell type may vary, depending on a variety of factors. The may include, for example, changes as a result of cellular proliferation, differentiation, disease, isolation of the cell from the body and culture in vitro. For example, cell surface proteins have been reported to play vital roles in multiple steps of prostate cancer metastasis, such as detachment of tumor cells from the extracellular matrix, resistance of tumor cells to the detachment-induced apoptosis, adhesion of tumor cells to endothelial cells and angiogenesis to support tumor growth. There may be cell surface proteins either uniquely or differentially expressed in both epithelial and endothelial cells that are important for individual processes of prostate cancer metastasis.

Researchers have taken advantage of the biological uniqueness of cell surface proteins and chemical properties of certain compounds to tag or “mark” cells. Cells marked in this manner can readily be isolated and characterized. In many cases, a combination of multiple markers is used to identify a particular cell type.

Antibodies are used extensively as diagnostic tools in a wide array of different analyses. Monoclonal and recombinant antibodies may be used as probes to tag or mark cells. Antibody-based immunoassays are the most commonly used type of diagnostic assay and still one of the fastest growing technologies for the analysis of biomolecules. Another example with particular importance in diagnostics that has become a routine during the past decade is flow cytometric analysis. Flow cytometry is a method for quantifying components or structural features of cells primarily by optical means. Although it makes measurements on one cell at a time, it can process thousands of cells in a few seconds. Since different cell types can be distinguished by quantifying structural features, flow cytometry can be used to count cells of different types in a mixture. Flow cytometers may also be employed to select or “sort” cells based on their cell surface marker expression.

Intracellular components may also be reported by fluorescent probes, including total DNA/cell (allowing cell cycle analysis), newly synthesized DNA, specific nucleotide sequences in DNA or mRNA, filamentous actin, and any structure for which an antibody is available. Flow cytometry can also monitor rapid changes in intracellular free calcium, membrane potential, pH, or cell signaling pathways. For example, Krutzik et al (Journal of Immunology, 2005, 175: 2357-2365) state “phosphospecific flow cytometry has emerged as a powerful tool to analyze intracellular signaling events in complex populations of cells because of its ability to simultaneously discriminate cell types based on surface marker expression and measure levels of intracellular phosphoproteins. This has provided novel insights into the cell- and pathway-specific nature of immune signaling.”

Flow cytometry analysis and sorting studies using monoclonal antibodies to define the surface markers on normal and neoplastic cell populations created the basis for routine clinical diagnostic assays that now range from leukemia classification to monitoring CD4 T-cell loss as HIV disease progresses.

Despite the versatility of flow cytometry, there are some drawbacks. For example, the antibodies chosen for flow cytometry reagents may not be optimal: They may not be specific, resulting in a high background staining. Furthermore, the efficiency of coupling the fluorochrome to the antibody may vary from batch to batch. Therefore, comparisons between populations of cells may be further complicated.

In addition, in order to assess multiple parameters on cell populations, a significant investment is required to purchase the required antibodies. For example, the analysis of 90 single parameters would require the purchase of 90 separate antibodies, which may amount to several tens of thousands of dollars and a large number of cells per analysis. This is problematic if the number of cells is finite and limited. Therefore, there is a significant need for cost effective, validated, reproducible reagents and antibodies for the phenotyping or analysis of cells.

In one example, Valet et al (Cytometry B,2003: 53B, 4-10) report the pretherapeutic identification of high-risk acute myeloid leukemia (AML) patients by data pattern analysis from flow cytometric immunophenotype, cytogenetic, and clinical data.

The interpretation of flow cytometry data can be subject to variation, attributed to factors such as, for example, variations between machines, and bias by the operator. Consequently, actual mean fluorescence intensities for a given marker on a given cell may vary greatly. Therefore, there is a significant need for a reliable automated method for analyzing flow cytometry data.

### SUMMARY

The present invention also provides a method for comparing the phenotypic state of a first unknown sample material with a sample material of a known phenotypic state. The method comprises the steps of:
a. Obtaining a first unknown sample material, and;
b. Contacting the first unknown sample material with reagents to detect the presence of phenotypic markers in the first unknown sample material, and;
c. Recording the presence of phenotypic markers in the first unknown sample material, and;
d. Obtaining a sample material of a known phenotypic state, and;
e. Contacting the sample material of a known phenotypic state with reagents to detect the presence of phenotypic markers in the sample material of a known phenotypic state, and;
f. Recording reagents the presence of phenotypic markers in the sample material of a known phenotypic state, and;
g. Reporting the differences between the unknown sample material and the sample material of a known phenotypic state.

The present invention provides a system for comparing the phenotypic state of a first unknown sample material with a sample material of a known phenotypic state. The system comprises:
a. A panel of phenotypic markers, and;
b. Reagents to detect the presence of phenotypic markers in the first sample material of unknown phenotypic state, and;
c. Apparatus to record the presence or absence of phenotypic markers in the first sample material of unknown phenotypic state, and;
d. Reagents to detect the presence of phenotypic markers in the sample material of a known phenotypic state, and;
e. Apparatus to record the presence or absence of phenotypic markers in the sample material of a known phenotypic state, and;
f. Apparatus to report differences between the first unknown sample material and the sample material of a known phenotypic state.

The present invention provides device for comparing the phenotypic state of a first unknown sample material with a sample material of a known phenotypic state. The device comprises:
a. A panel of phenotypic markers, and;
b. Reagents to detect the presence of phenotypic markers in the first sample material of unknown phenotypic state, and;
c. Apparatus to record the presence or absence of phenotypic markers in the first sample material of unknown phenotypic state, and;
d. Reagents to detect the presence of phenotypic markers in the sample material of a known phenotypic state, and;
e. Apparatus to record the presence or absence of phenotypic markers in the sample material of a known phenotypic state, and;
f. Apparatus to report differences between the first unknown sample material and the sample material of a known phenotypic state.

In one embodiment, the apparatus that reports the differences between the first unknown sample material and the sample material of a known phenotypic state is a computer running a statistical analysis program. In one embodiment, the statistical analysis program is a modified Kolmogorov-Smirnov two-sample test.

The present invention provides kit for comparing the phenotypic state of a first unknown sample material with a sample material of a known phenotypic state. The kit comprises:
a. A panel of phenotypic markers, and;
b. Reagents to detect the presence of phenotypic markers in the sample materials.

In one embodiment, the phenotypic state of a first unknown sample material is tissue or cells obtained from a diseased patient. The sample material of a known phenotypic state is tissue or cells obtained from a healthy patient.

In one embodiment, the phenotypic state of a first unknown sample material is tissue or cells obtained from a patient treated with a drug. The sample material of a known phenotypic state is tissue or cells obtained from a patient not treated with a drug.

In one embodiment, the phenotypic state of a first unknown sample material is tissue or cells obtained from a patient after the patient is treated with a drug. The sample material of a known phenotypic state is tissue or cells obtained from a patient before the patient is treated with a drug.

These embodiments and more are described more fully in the following sections.

### BRIEF DESCRIPTION OF THE DRAWINGS

FIG. 1: Outline of the method of the present invention.

FIG. 2: Analysis of the phenotypic state of a population of cells using the marker reagent array shown in Table 2.

FIG. 3: Analysis on the effects of enzymatic treatment on the phenotypic state of different samples of populations of cell X, using the marker reagent array shown in Table 2.

FIG. 4: Comparison of the phenotypic state of a sample of cell X with a sample of human dermal fibroblasts, using the marker reagent array shown in Table 2.

FIG. 5: Phenotypic analysis of whole blood. FIG. 5A shows the level of expression of a selected epitope in heterogeneous cell samples. Leukocyte, monocyte and granulocyte populations were selected based upon size and granularity. Percentage of positive events and Mean Fluorescence Intensity was assessed for all selected cell populations for each epitope tested. As an example the expression levels of CD63 are shown for all selected cell populations. FIG. 5B shows the percentage of positive events and expression levels of leukocytes, monocytes and granulocytes for the array of surface epitopes shown. Cell populations were selected and analyzed from three healthy donors in 11 separate tests (Mean±SEM). FIG. 5C shows expression levels of the epitopes in the reagent array shown in table 1 for a leukocyte population taken from a healthy individual.

FIG. 6: Gating strategy for homogeneous (A) and heterogeneous (B) samples. (A). Events were acquired until 10.000 events were counted in the gate with a maximum of 100 s. (B). Lymphocytes and monocyte populations were individually gated. Erythrocytes and debris were removed from the scatter by increasing the threshold. Events were acquired until 10.000 events were counted in the lymphocyte gates with a maximum of 100 s.

FIG. 7: Phenotypic state of a human umbilical cord derived cell-line (UTC) as determined by flow cytometry. Phenotypic state of human UTCs (n=8). Data expressed as geom. Mean±Standard error of the mean (SEM). Markers with MFIs less than 25 are considered low expressed. Markers with MFIs between 25 and 75 are considered intermediate expressed and MFIs greater than 75 are considered to be highly expressed.

FIG. 8: The influence of the dissociation enzymes Trypsin, Trple and Accutase on the phenotypic state of hUTCs. Dissociation enzymes affect the expression of several markers (circled). Red circles were at least 1 standard deviation form the average of the training set and also found different with the modified Kolmogorov-Smirnov test used in the present invention. Black circles were less than 1 standard deviation from the average of the training set and identified as different by the modified Kolmogorov-Smirnov test or more than 2 standard deviations from the average and not identified as different by the modified Kolmogorov-Smirnov test.

FIG. 9: Analysis of the influence of different dissociation enzymes on the phenotypic state of UTCs with the modified Kolmogorov-Smirnov test used in the present invention. The red colored squares are considered significantly different from the other samples. In the upper figure all datasets are analyzed individually and unbiased. In the lower figure the datasets of the dissociation enzyme-harvested cells are compared to a training set (n=8).

FIG. 10: The phenotypic state of PBMCs isolated with Ficoll-Hypaque gradient centrifugation, as determined by flow cytometry. Upper: corrected MFIs for Lymphocytes (left) and Monocytes (right). Lower: percentage of positive cells for Lymphocytes (left) and monocytes (right). Data expressed as mean±SEM.

FIG. 11: Phenotypic state of lymphocytes (A) and monocytes (B) isolated with Ficoll-Hypaque and BD cell preparation tubes (CPT), as determined by flow cytometry. The expression of surface markers of PBMCs isolated with Ficoll-Hypaque (▪) (n=13) were compared to PBMCs isolated with cell preparation tubes (▴) (n=5). Differences between the two methods of isolation were small and both methods cause similar variation. Data expressed as mean±SD.

FIG. 12: Phenotypic state of lymphocytes (A) and monocytes (B) for 90 markers. A marker with an MFI of less than 25 is considered negative or low expressed. Markers with MFIs between 25 and 75 (indicated with dotted lines) are intermediately expressed. Markers with MFIs above 75 are highly expressed. Data expressed as mean±SD.

FIG. 13: Distribution of standard deviations per donor. The left panel shows the SD distribution of lymphocytes. The right shows the SD distributions of monocytes. For quality control the MFI values were expressed as standard deviations from the average.

FIG. 14: Distribution of standard deviations per marker. The MFI values were expressed as standard deviations from the average sorted by marker. The left panel shows the SD distribution of the lymphocytes, the right panel shows the distribution of the monocytes.

### DETAILED DESCRIPTION

According to the present invention, assay components and methods for the characterization of the phenotype of cells are provided. As those of ordinary skill in the art will recognize, the invention has an enormous number of applications in diagnostic assay techniques. Reagents may be prepared, for example, so as to detect or screen for any of a number of sample characteristics, pathological conditions, or reactants. The present invention provides panels of reagents that are selected to cover various applications.

Suitable panels of reagents may include, for example, a tumor marker panel including epitopes related to prostate cancer, breast cancer, hematological disorders, and other suitable tumor markers; an autoimmune disease panel comprising tests for rheumatoid factors and other markers associated with autoimmune disease; a therapeutic drug panel comprising tests for biomarkers related to the drug therapy of interest.

### The Reagents

Reagents suitable for use in the present invention may be antibodies that recognize phenotypic markers. The phenotypic markers may be proteins expressed on the surface of cells, or they may be proteins that are expressed intracellularly. Alternatively, they may be proteins that are secreted from the cell. Alternatively, the phenotypic markers may be nucleic acids, lipids, polysaccharides or any biomolecule.

Reagents suitable for use in the present invention may be antibodies that specifically the phenotypic markers. Alternatively, the reagents may be oligonucleotides or any molecule that specifically recognize the phenotypic marker. In one embodiment, the reagents are labeled with a fluorescent marker. Alternatively the reagent may be intrinsically fluorescent.

Antibodies suitable for use in the present invention may be obtained from commercial sources, or they may be generated for a specific application. In one embodiment, the antibodies are labeled with a fluorescent marker. Examples of antibodies and phenotypic markers suitable for use in the present invention are shown in Table 1.

Antibodies may be selected that represent phenotypic markers of a state of interest or can be selected randomly. For example, the phenotypic markers may be proteins that are typically expressed on stem cells. Alternatively, the phenotypic markers may be associated with a disease, such as, for example, cancer. Alternatively, the phenotypic markers may be used to measure molecules in heterogeneous systems, such as, for example, blood.

### The Reagent Panels

The present invention provides cost effective, optimized reagents for the reproducible analysis of cells. In one embodiment, the reagent is in the form of a kit, comprising a multi-well plate containing antibodies that are lyophilized or otherwise configured allowing for convenient transport and storage. The multi-well plates may have different formats such as 6, or 8, or 12, or 24, or 96, or 384 wells and the like. The antibodies are selected according to the analysis that is to be performed. The antibodies may be selected to cover immune related epitopes. Alternatively, they may be selected to cover cancer epitopes, or stem cell epitopes. The antibodies may recognize extracellular molecules or they may recognize intracellular molecules.

The selected antibodies may be dispensed into multi-well plates according to the layouts shown in Tables 2 and 3. The layouts are unique to the specific phenotypic marker panel. Each well of the multiwell plate may contain a single antibody or multiple antibodies. The selected antibodies are provided in bulk quantities and dispensed into a large number of plates to reduce variation between samples. The antibodies may be stored in the plates in solution prior to analysis. Alternatively, the antibodies may be stored lyophilized and re-constituted prior to analysis.

### The Method

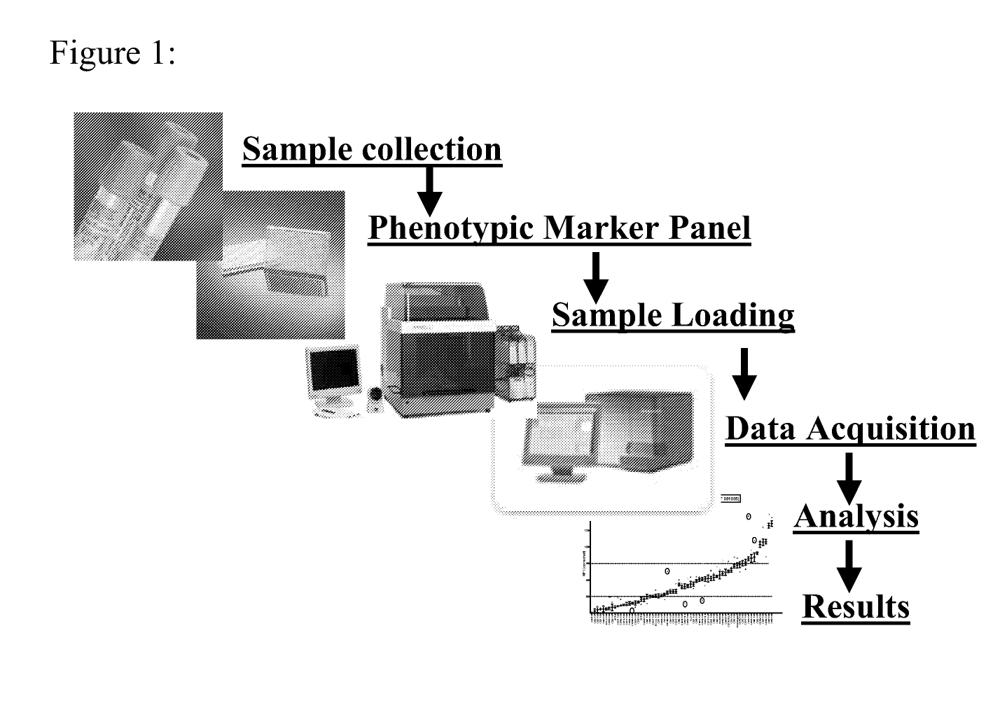

In one embodiment, the method of the present invention comprises a system that carries out the steps of collecting samples material of an unknown phenotypic state and collecting sample material of a known phenotypic state. The sample material may comprise, for example, cells, blood, or any biological sample. The system analyses the sample materials to identify patterns epitope markers in the sample material of known phenotypic state that is used as a comparator for the sample material of unknown phenotypic state. The identity of the epitopes in the identified pattern may be known. Alternatively, more than one epitope in the identified pattern may be unknown. An outline of the method of the present invention is shown in FIG. 1.

For example, the methods of the present invention may be used for quality control purposes. In this case, the sample material of known phenotypic state may be obtained from a control population of cells. This sample material may then be used as a comparator for the sample material of unknown phenotypic state that consists of populations of cell obtained from production runs. Changes from the patterns of epitope markers in the sample material of known phenotypic may be used to accept or reject production runs. An example of this is shown in Example 1.

Alternatively, the present invention may be used to report changes in the pattern of epitope markers in a population of cells following treatment with a factor or pharmaceutical agent. An example of this is shown in Example 2. Alternatively, the present invention may be used to report changes in the pattern of epitope markers between distinct populations of cells. An example of this is shown in Example 3.

In one embodiment, the present invention may also report changes in the pattern of epitope markers in distinct populations of cells in whole blood. The cell populations may be from a sample of blood from one patient. Alternatively, samples from more patient may be used.

The methods of the present invention may be employed to analyze one sample or, alternatively, more than one sample. In one embodiment, the high-throughput analysis of a large number of samples may be carried out. The steps of the method of the present invention may be carried manually. Alternatively, at least one step may be automated. Multiple copies of a phenotypic marker reagent plate may be made and used to analyze samples.

Analysis of the samples may be performed by any suitable platform known to those of ordinary skill in the art. Such platforms may include, for example, a flow cytometry apparatus, a mass spectrometry apparatus, or a fluorescent microscope, and the like.

Flow cytometry apparatuses rely upon flow of cells or other particles in a liquid flow stream in order to determine one or more characteristics of the cells under investigation. Further, the flow cytometry apparatus is useful for identifying the presence of certain cells or particles of interest, enumerating those cells or particles and, in some instances, providing a sorting capability so as to be able to collect those cells or particles of interest. In a typical flow cytometry apparatus, a fluid sample containing cells is directed through the flow cytometry apparatus in a rapidly moving liquid stream so that each cell passes serially, and substantially one at a time, through a sensing region. Cell volume may be determined by changes in electrical impedance as each cell passes through the sensing region. Similarly, if an incident beam of light is directed at the sensing region, the passing cells scatter such light as they pass there through. This scattered light serves as a function of cell shape and size, index of refraction, opacity, roughness and the like. Further, fluorescence emitted by labeled cells, or autofluorescent cells, which have been excited as a result of passing through the excitation energy of the incident light beam is detectable for identification of cells having fluorescent properties. After cell analysis is performed by the flow cytometry apparatus, those cells that have been identified as having the desired properties may be sorted if the apparatus has been designed with such capability.

Representative flow cytometry apparatuses are described in U.S. Pat. Nos. 3,826,364 and 4,284,412, and in the publication by Herzenberg et al., “Fluorescence-activated Cell Sorting,” Sci. Am. 234 (3): 108, 1976.

### Cytomics

The methods of the present invention are utilized to profile the differences between phenotypic states of samples. The samples may be, for example, samples of tissues or cells that are obtained from a patient with a given disease, and samples from a patient who does not have the disease. The methods of the present invention will determine differences in the phenotypic state between the two samples. Alternatively, the samples may be obtained from a patient treated with a drug and samples from a patient not treated with a drug. Alternatively, samples can be taken from a single patient before and after drug treatment. The methods of the present invention will determine differences in the phenotypic state between the two samples.

Data, corresponding to the differences between the phenotypic states of the samples can be generated by applying analytical techniques to samples of body fluids, cells or tissues obtained from in vivo studies. The range of data that can be generated in an investigation of a disease or of a drug response can extend from a dataset created by applying, to a single cell type, a single analytical platform that focuses on a single class of molecules (for example, RNAs or triglycerides). Alternatively, data can be obtained from the analysis of samples from multiple tissues and body fluids with an array of analytical platforms that can capture as many biochemical changes as technically possible. Data can reflect the comparison of just two ‘stable’ states of the system or the dynamics of a transient response to a drug treatment or of the progression of a disease. Representative methods for the generation of such data are described in Van de Greef & McBurney (Nature Reviews Drug Discovery 4, 961-967).

In one embodiment, data, corresponding to changes in phenotypic state of two samples are generated by high throughput flow cytometry, using the methods of the present invention (see example 5).

### Data Analysis

In one embodiment, the present invention utilizes a modified Kolmogorov-Smirnov test to report changes in the phenotypic state between two samples. The Kolmogorov-Smirnov two-sample (KS) test is a statistical test used to determine if the distribution of events of a parameter from two samples arises from the same underlying unknown distribution. The Kolmogorov-Smirnov test is a non-parametric test that is capable of identifying differences in location and shape of the distribution. The parameter tested may be, for example the presence of a cell surface marker, as detected by flow cytometry.

Given a set of n samples, {Xᵢ}ᵢ₌₁ⁿ, each sample, Xᵢ, is a r×s matrix, where r is the number of events recorded, s the number of parameters in the experiment and each element of the matrix, x_{i,j,k}, is the fluorescent intensity value (x_{i,j,k}ε[0, C−1], C the number of channels). All the k-th column values of a sample can be organized into a 1×r column vector, P_{i,k}=[x_{i,1,k} x_{i,2,k} . . . x_{i,r,k}]^{T}, known as the parameter vector. Each sample can be represented as:

$${X}_{i} = [ \begin{matrix}{x}_{i , 1 , 1} & {x}_{i , 1 , 2} & ⋯ & {x}_{i , 1 , s} \\ {x}_{i , 2 , 1} & {x}_{i , 2 , 2} & ⋯ & {x}_{i , 2 , s} \\ ⋮ & ⋮ & ⋰ & ⋮ \\ {x}_{i , r , 1} & {x}_{i , r , 2} & ⋯ & {x}_{i , r , s}\end{matrix} ] = [ \begin{matrix}{P}_{i , 1} & {P}_{i , 2} & … & {P}_{i , s} ]\end{matrix} \qquad ( 1 )$$

A class label to each sample is assigned, denoted by yᵢ, where yᵢε{+1 −1}. The set of all class labels can be organized into a 1×n class vector, Y=[y₁ y₂ . . . yₙ], with n⁺ the number of samples from the positive class (yᵢ=+1) and n⁻ the number of samples from the negative class (yᵢ=−1).

Given two parameter vectors for the k-th parameter, P_{a,k} and P_{b,k}, the D-value (D_{a,b}) in a Kolmogorov-Smirnov two-sample test is calculated as
D_{a,b}(k)=max(|F(P_{a,k})−F(P_{b,k})|) (2)
where F(P_{i,k}) is the cumulative distribution function of all the events in the parameter vector P_{i,k}. Based on a significance level (α), the critical D-value for the k-th parameter (\hat{D}ₖ), is proportional to

$$\sqrt{\frac{1}{r}} ,$$

where r is the number of events in the sample. If the D_{a,b} is greater than this critical D-value, the distribution of events in the two parameter vectors as said to be significantly different at a level of α. The D-value is determined empirically for each parameter. The critical D-value (\hat{D}ₖ) is the maximum D-value obtained when comparing the samples from the negative class to each other and samples from the positive class with each other. The methods of the present invention contain a modification to the Kolmogorov-Smirnov test, where a threshold is placed on the data to determine the level of significance of the data. Mathematically,
\hat{D}ₖ=max(D_{a,b}(k),D_{p,q}(k)
yₐ,y_{b}ε+1 yₚ,y_{q}ε−1 (3)

Next, the D-values obtained by comparing the k-th parameter distribution between samples from the negative class (y=−1) to samples from the positive class (y=+1) are recorded. The k-th parameter is said to have a different distribution between the two classes if more than 90% of the D-values recorded are greater than the critical D-value (\hat{D}ₖ) for that parameter.

Hierarchical clustering analysis of the D-values obtained for the parameters of each sample, determines which parameters are shared or differ between the samples tested. In one embodiment, a Robust Competitive Agglomeration Algorithm (for example, as disclosed in (Krishnapuram, H. F. a. R., IEEE Transactions on Pattern Analysis, 1999. 21(5): p. 450-465) is used to perform the clustering analysis.

The present invention is further illustrated, but not limited by, the following examples.

### EXAMPLES

### Example 1

### Quality Control; Release Criteria

Master plate preparation: An antibody template was designed to monitor changes of expression across a wide array of epitopes in order to establish consistency of the cell product between production and expansion runs. Antibodies were selected and dispensed into a multiwell plate according to the layout shown in Table 2. Antibodies were purchased from the sources shown in Table 1. Antibodies were diluted in PBS into a 96-well master plate and stored in a refrigerator prior to analysis. For each assay 10 μl of master antibody solution was transferred from the master plate to a new 96 well plate as such that a copy was created. The array contained phycoerythrin fluorescein labeled antibodies against one specific marker per well. This eliminated the need for compensation of the machine, simplified the assay and reduced operator bias. In addition, the staining protocol was designed to minimize the number of cells needed for the assay and eliminate steps, such as washing, that can cause errors.

Assay: A vial of cells were thawed, washed and finally diluted to 2×10⁵ cells per ml in PBS and a 100 μl sample was transferred to each well (20,000 cells/well) containing diluted antibodies. The plate was left to incubate for 30 minutes at 4° C. Next 100 μl fixation buffer (Cytofix, BD catalog no. 554655) was added and left for 15 minutes. A FACSCalibur machine with a high through put system was setup for analysis and 10,000 events per well were acquired.

Data analysis: For this study the acquired data was exported to an analysis software program and the Mean Fluorescent Intensity (MFI) of each sample well was assessed. The MFI of the unstained sample was subtracted from each sample MFI and this delta MFI was plotted for each epitope. This was repeated eight times in independent experiments creating an average expression pattern for our cell product.

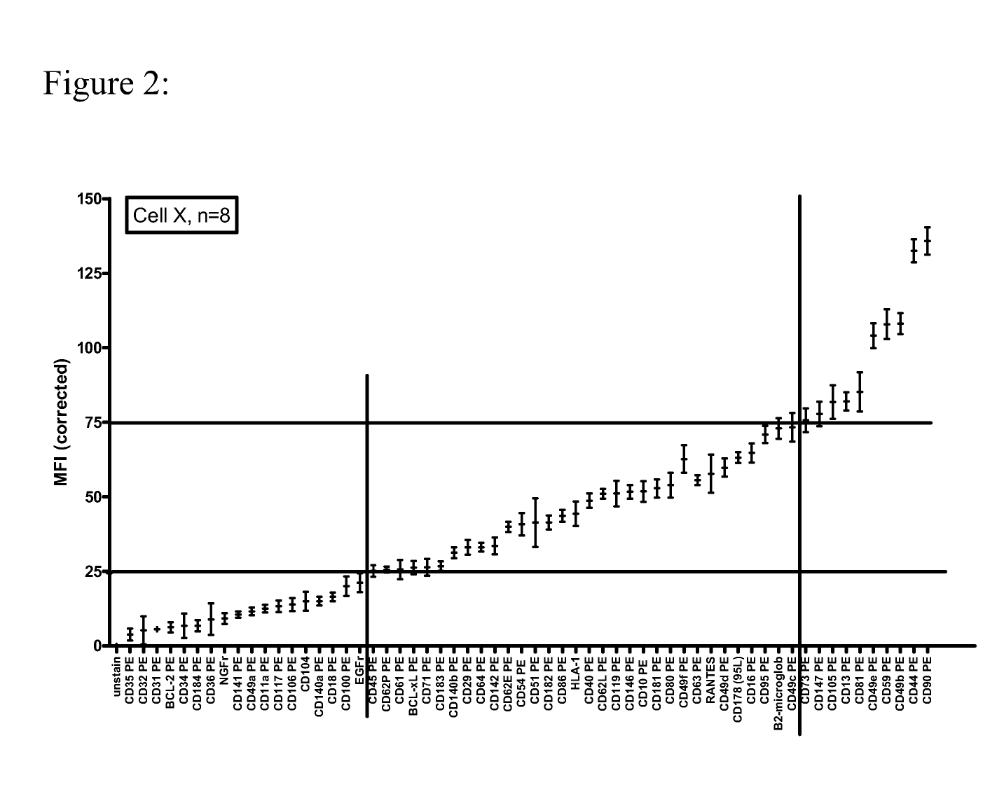

Results: Cells obtained from different production/expansion runs were routinely stained using the 96-well plate array and the MFI were plotted and compared against the pattern of epitope markers obtained from the sample material of known phenotypic state. The results are shown in FIG. 2.

### Example 2

### Comparison of Dissociation Reagents

Method: The methods and reagent panel used for this assay is the same as described in Example 1. Cells were grown in 3 separate culture flasks under identical conditions. These cultures were harvested each with a different dissociation enzyme mix. The cells were then stained using out 96-well method and the mean fluorescence intensity was measured for each parameter and compared to the pattern of epitope markers obtained from the sample material of known phenotypic state.

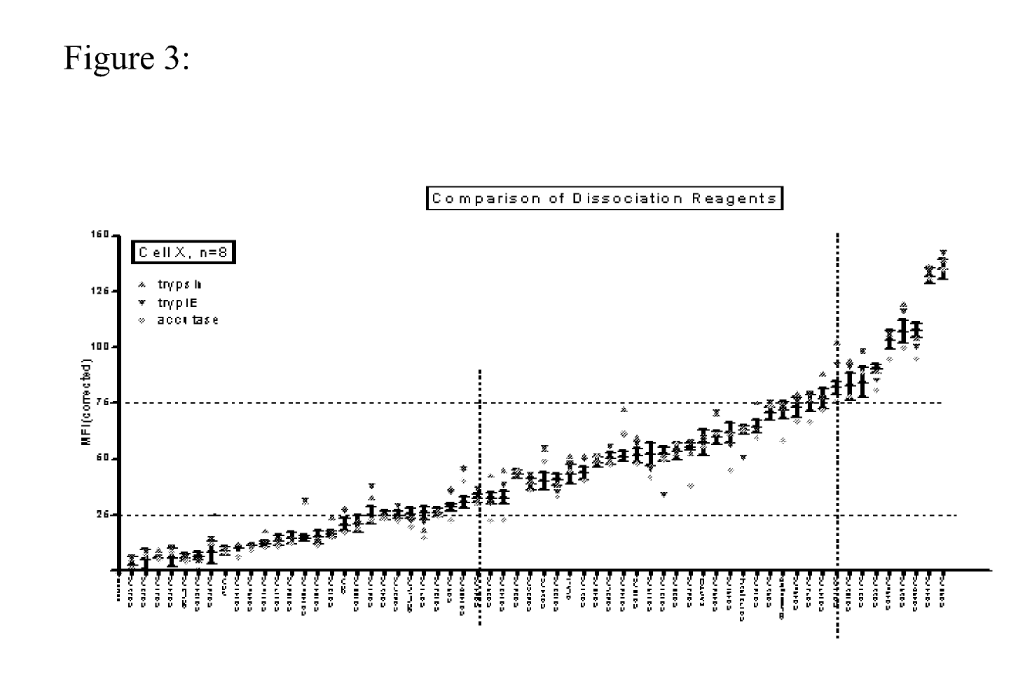

Results: Each enzyme mix showed different results when compared to the pattern of epitope markers obtained from the sample material of known phenotypic state. Mixture C (Accutase) seems to affect the expression of certain phenotypic marker more than the standard mix A (Trypsin) and B (TriplE). For example the adhesion marker CD49f and the signaling molecule CD63 show lower expression levels after the harvesting procedure, which may affect the cell product's function. Results are shown in FIG. 3.

Conclusion: This method allows for a rapid assessment of a wide array of proteins expressed on the cell surface. Changes to the phenotypic state that may occur following treatment with enzymes can readily be assessed by this method. The availability of a master plate allows for a quick assessment of phenotypic changes across a wide array of phenotypic markers due to treatment of the cells.

### Example 3

### Comparison of the Phenotypic State of Two Distinct Cell Populations

Method: The methods and reagent panel used for this assay were the same as described in Example 1. In this assay expression levels epitopes tested by the master plate on a different homogeneous cell culture. The cell culture of interest was grown to confluency and harvested according our standard protocol. Both cell cultures were then stained in the 96-well master plate and analyzed according the methods described earlier. The phenotypic state of the cell populations were compared and key differences were identified.

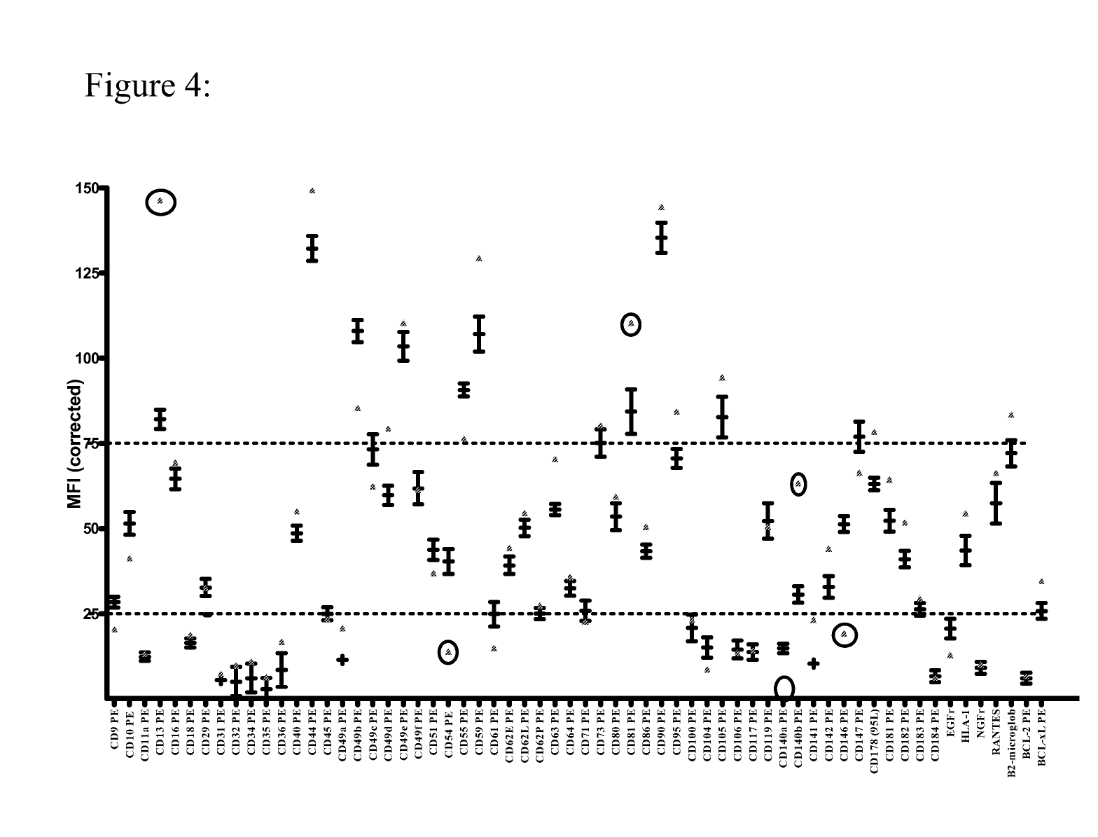

Results: The two cell types have similar expression levels for most of the epitopes except for example CD11a and CD54. These epitopes were differentially expressed. We selected an arbitrary cut-off for significance at a delta of 25 MFI compared to the sample material of known phenotypic state. The results are shown in FIG. 4.

Conclusion: We showed expression differences between two cell types in our panel. These differences can be further explored and can become important starting points of further research especially if the phenotypic marker changes can be linked to in-vivo efficacy differences.

### Example 4

### Phenotypic Analysis of Whole Blood

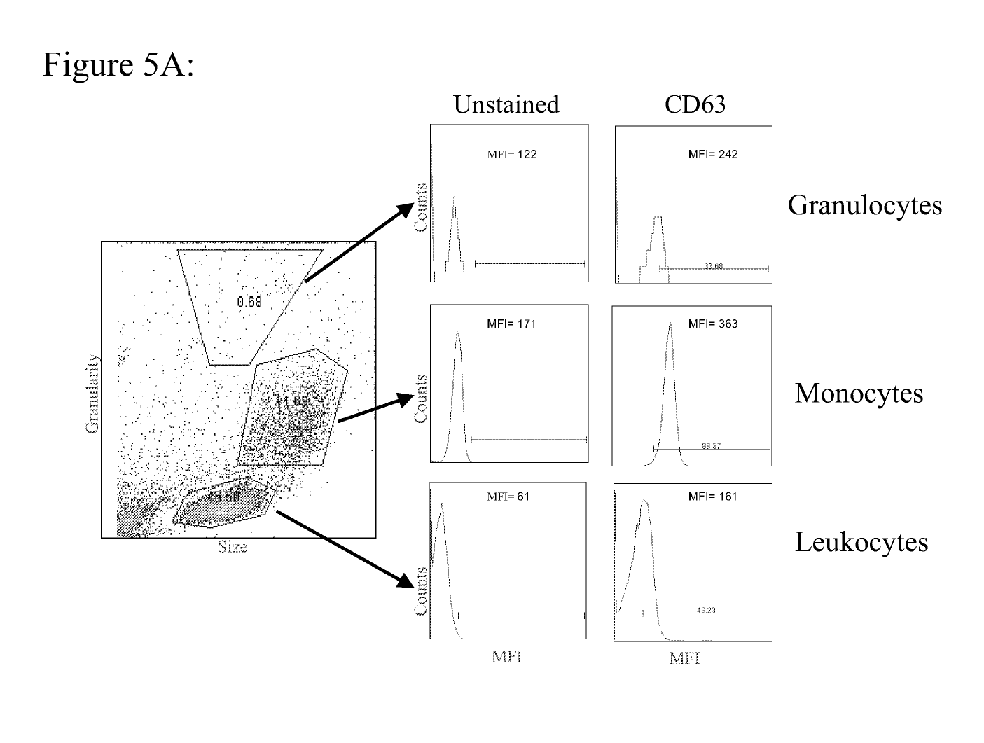

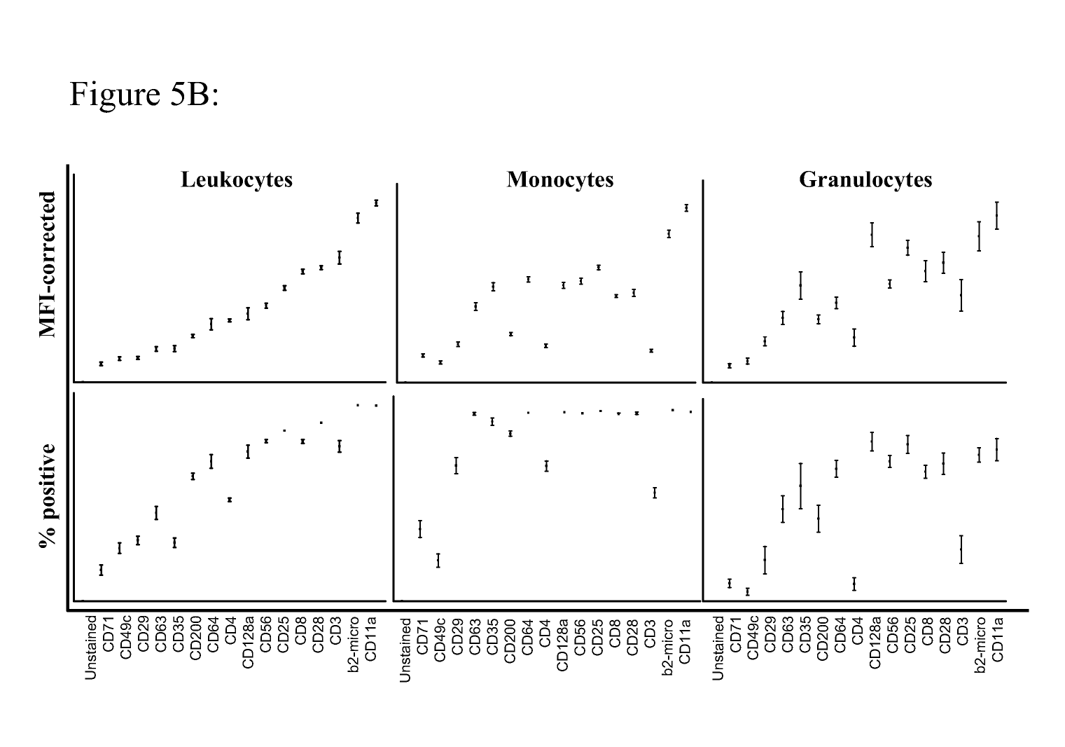

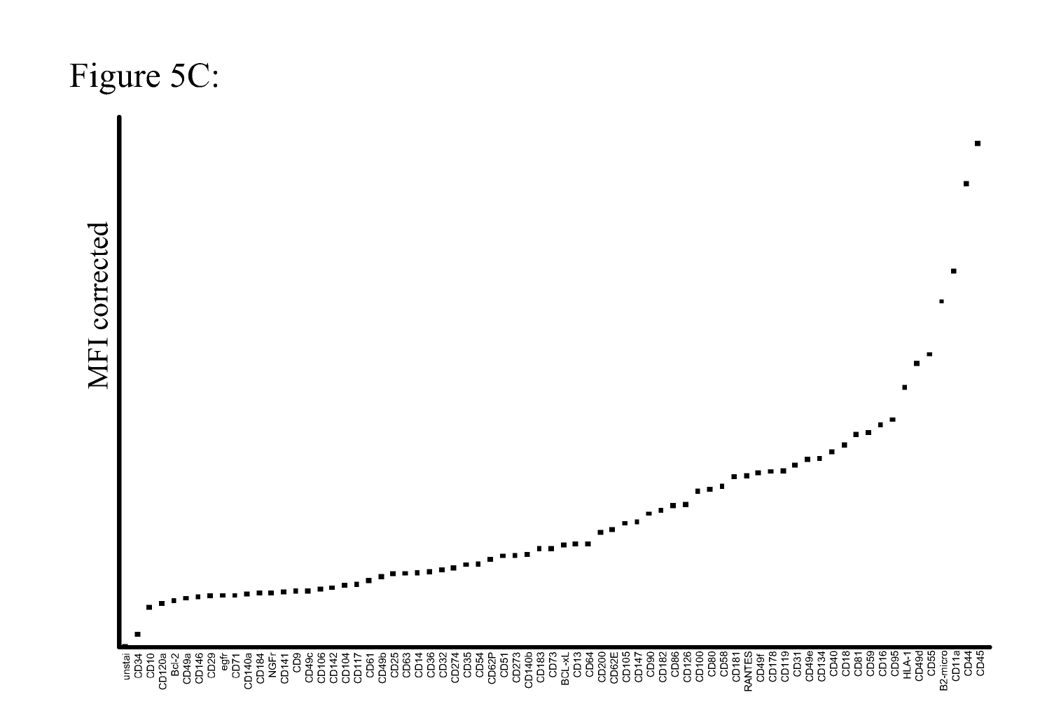

Method: The methods and reagent panel used for this assay were the same as described in Example 1. Heparinized peripheral blood was obtained from healthy volunteers and white blood cells were isolated. The White blood cells were washed and suspended in PBS and transferred to the 96-well staining plate (50,000 cells per well) and data was acquired according to the standard protocol. Data analysis, however, is different since the sample contains multiple cell populations each with their specific expression pattern for each epitope. Analyzing the cells as a bulk may obscure these effects. Therefore three main populations were selected based upon size and granularity and each was analyzed for percentage positive events within the gate and expression levels of each epitope tested (FIGS. 5A,B and C).

Results: Using the 96-well platform we able to quickly assess expression levels of our array of epitopes for three distinct populations in peripheral blood (FIG. 5).

Conclusion: The 96 well plate can also be used for heterogeneous cell population. However the data processing and mining becomes significantly more complex. Novel analysis methods are in development to simplify the analysis, which is especially important when this method is expanded to multiple color flow cytometry. This method may be used to determine the pattern of epitope markers in easy accessible peripheral blood samples for healthy (non-disease phenotype) individuals and use this a comparator for cytomes obtained from individuals with a particular disease. Differences can be exploited for, but not limited to, development of diagnostics, measure drug efficacy and selection of treatment regiment.

### Example 5

### High Throughput Immunophenotyping

Methods:

This example shows the development of a method for the high throughput immunophenotyping of homogeneous and heterogeneous cell populations, based on flow cytometry. A method for high throughput immunophenotyping homogeneous cell types may be used as a basic research tool to measure cell surface protein expression. A method for high throughput immunophenotyping heterogeneous cell populations can be used for phenotyping peripheral blood mononuclear cells (PBMCs). PBMCs are hypothesized to interact with all other types of cells. These interactions will induce changes in the phenotypes of these cells. Phenotyping PBMCs therefore has many applications.

To develop a method for high throughput immunophenotyping cell populations several challenges must be dealt with. Currently, methods of analyzing flow cytometric data are subject to the bias of the experimenter. In addition, high throughput immunophenotyping will provide large amounts of data which complicates analysis.

A fully automated method for high throughput immunophenotyping cell populations in combination with methods for analyzing large amounts of flow cytometric data is a promising technique in diagnosis.

Homogeneous cell populations: Cryopreserved M21 human melanoma cells, kindly provided by Dr. Frieder Kern (DKFZ, Germany), Huvec human umbilical veil endothelial cells (Huvec), PC-3 and DU-145 human prostate cancer cells and BT-20, MCF-7 and MDA-MB-231 human breast cancer cells were all kindly provided by Celia Sharp (Centocor R&D, USA). The cells were thawed and transferred to PBS. The cells were centrifuged at 250 RCF for 3 minutes, resuspended in PBS, counted (Vi-cell XR™, Beckman Coulter), resuspended in PBS (200.000 cells/ml) and immediately used for analysis.

Blood samples and PBMC isolation: Two methods were used to process blood and isolate mononuclear cells: Ficoll-hypaque gradient centrifugation and Cell preparation tubes (CPT) also based on Ficoll-hypaque. For both methods 8-10 ml of venous whole blood was collected from healthy volunteers in heparinized tubes and processed within one hour of collection.

In the first method, Ficoll-hypaque gradient centrifugation, blood samples were diluted 1:2 with PBS. 10 ml Leukocyte Separation Medium (LSM, MP biomedicals) was underlayed. Subsequently, the samples were centrifuged for 20 min at 500 RCF and the PBMCs were harvested from the interface. PBMCs were washed in PBS and erythrocytes were lysed with 2-5 ml ACK lysing buffer (Cambrex) for 2 minutes. The cells were resuspended in PBS and counted (Vi-cell XR™, Beckman Coulter).

In the second method heparinized BD vacutainer® CPT™ Cell Preparation Tubes were used according to manufacturers instructions. The tubes were centrifuged 15 minutes at 1500 RCF, after which the upper-half of the plasma layer was discarded. The remainder of the plasma layer and the PBMC layer were collected, washed and resuspended in PBS and counted (Vi-cell XR™, Beckman Coulter). Subsequently, the cell concentration was adjusted to 0.5 106 cells/ml and the cell suspension was ready for immediate usage.

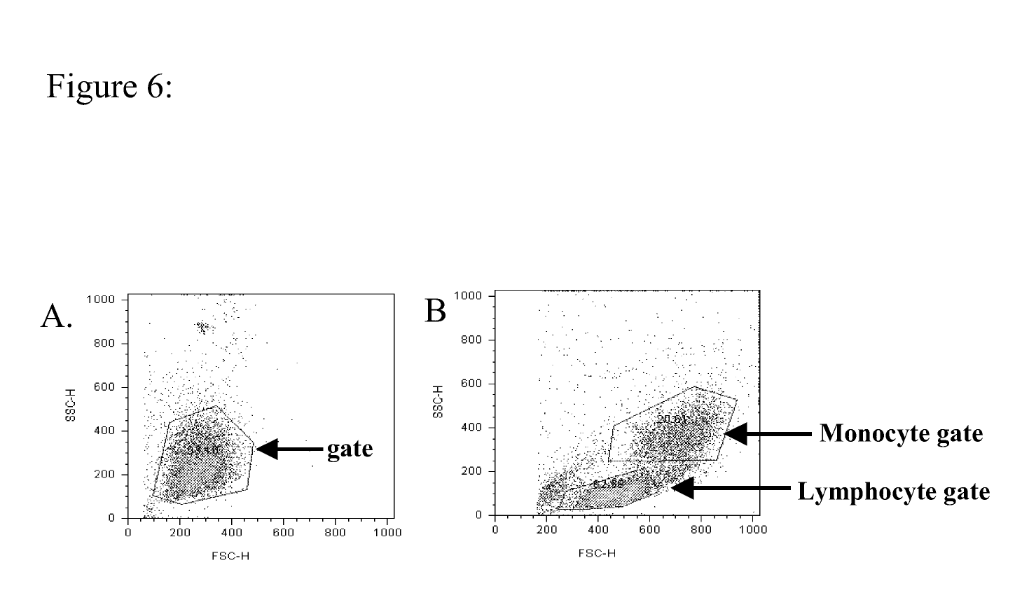

Flow cytometry: 96-well plates were filled with phycoerythrin (PE) conjugated antibodies directed against cell surface markers (Table 4 A&B) and 20-50 μl/106 cells in predefined plate layouts. Table 5 shows the 17 markers used for comparing the different methods. Table 6 shows the markers used for immunophenotyping PBMCs and the proof of principle experiments with the various cancer cell lines. The unstained wells A1-A5 are to set up the instrument. 20.000 cells for the homogeneous cell samples or 50.000 PBMCs were added per well and incubated for 30 minutes at 4° C. 100 μl BD cytofix buffer (BD Biosciences) was added and the cells were stored at 4° C. until analysis, but at least 15 minutes. For Flow cytometric analysis a FACSCalibur™ with the High-throughput sampler (HTS) for 96-well plates was used (BD Biosciences). For PBMC samples a threshold was set to exclude debris and erythrocytes. For analysis FlowJo® software (Tree Star, Inc.) was used. Homogeneous cell populations were gated as shown in FIG. 6, panel a. Lymphocytes and monocytes were gated as shown in FIG. 6, panel b. Events were acquired until 10.000 events were counted within the gate with a maximum of 100 seconds. For PBMCs 10.000 events within the lymphocyte gate were counted.

Data analysis: The geometrical Mean Fluorescence Intensity (MFI) was corrected for background and a specific staining by subtracting the MFI of an unstained sample. The geometrical mean is the antilogarithm mean of the logged MFI values. Geometrical (geom.) means were preferred over the normal mean PE Fluorescence. In addition, a statistical pattern recognition approach, based on the Kolmogorov-Smirnov two-sample test and Neyman-Pearson Kolmogorov-Smirnov test, was used to identify differences between samples. In short, probability contribution functions (pcf) were calculated for each individual run and for all parameters and used to analyze cumulative distribution functions (cdf). The largest difference in the cdf (Dmax) is obtained for all parameters of each run and expressed in a matrix.

Results:

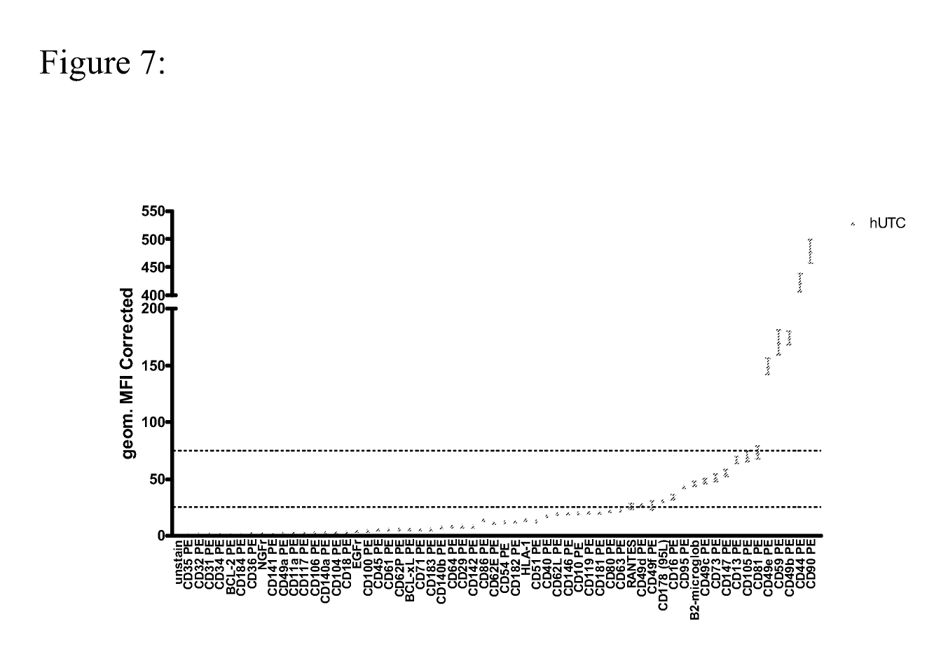

Homogeneous cell populations: A method for immunophenotyping of homogeneous cell populations was developed in the summer of 2005 by Kate Grandfield (SCIV, USA). As an example the phenotyping results of human Umbilical cord derived cells are shown in FIG. 7.

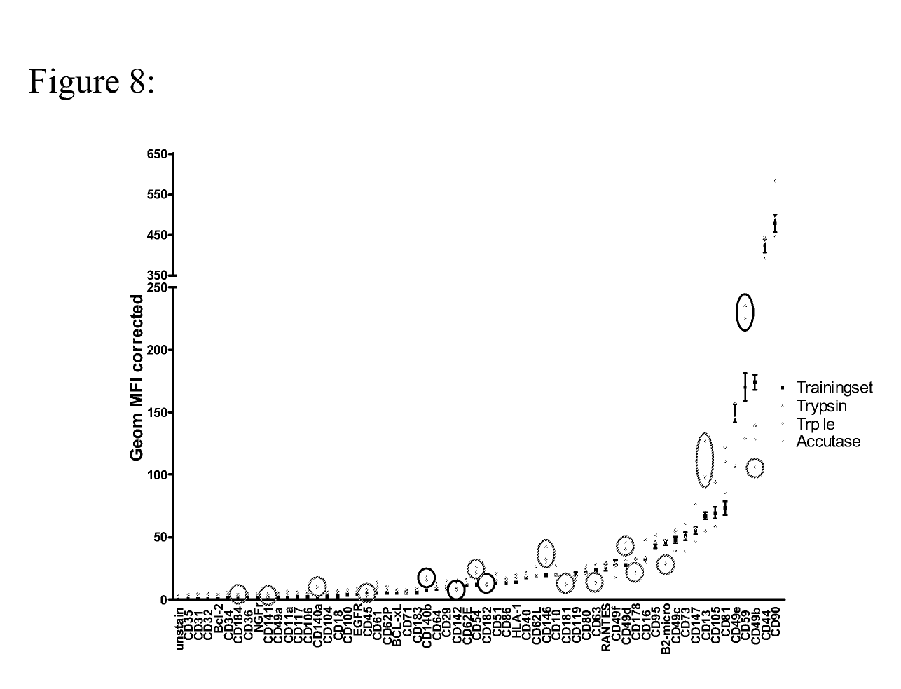

Data analysis: When more parameters are tested and more cell types, data analysis rapidly becomes more complicated. To overcome these complexities the Kolmogorov-Smirnov test was applied to identify differences in MFIs. To test the method it was applied on data of an experiment testing the influence of three different dissociation enzymes, Accutase, Trypsin and Trp Le, on the phenotype of UTCs (Data obtained from Jeffrey Kennedy, SCIV, 2005). The enzymes were compared to the phenotype of the training set as shown in FIG. 7 (n=8). The results of this experiment are shown in FIG. 8.

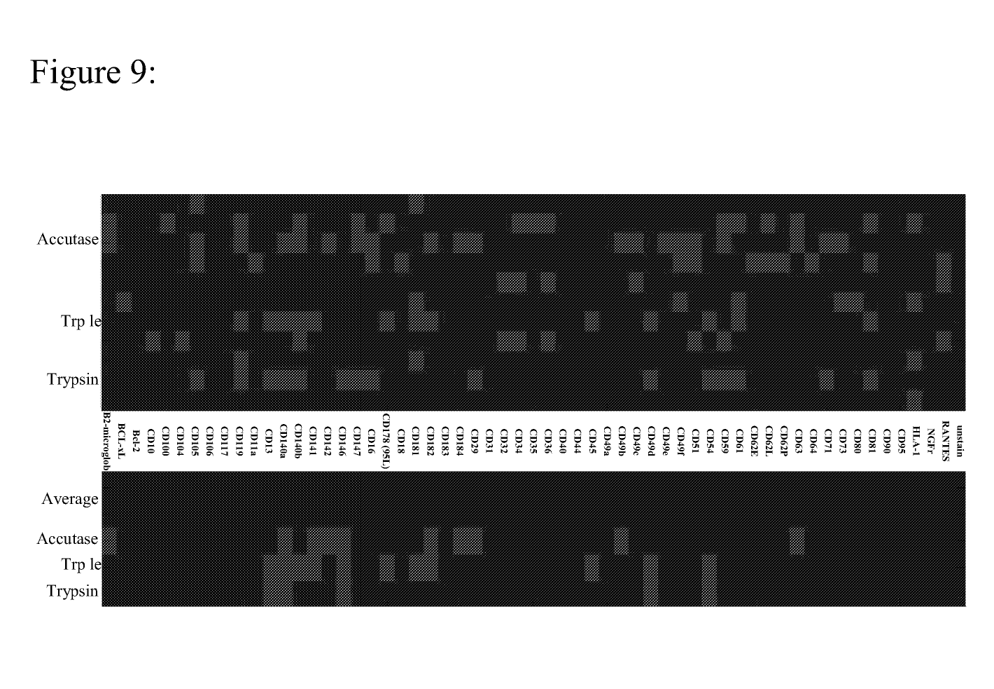

FIG. 9 shows the same data now analyzed with the Kolmogorov-Smirnov test. The upper figure represents the datasets of the training set (n=8) and UTCs harvested with the dissociation enzymes, all analyzed individually and unbiased. Red colored squares are considered significantly different from the other samples.

The methods of the current invention can also be applied to screen for a known disease. In this case an unknown sample will be compared to a training set of healthy subjects. The lower part of FIG. 9 shows the results when the Kolmogorov test is applied to the same data when the influence of the dissociation enzymes is compared to a training set (n=8).

The experiments with the dissociation enzymes were performed only once (n=1), which will not allow testing for significance. Therefore, samples that were 2 standard deviations (SD) from the training set average were considered significantly different. Because of increased sensitivity, application of the Kolmogorov-Smirnov test on the individual samples identifies more samples as different, including samples that differ between 1 and 2 SD of the average of the training set. The Kolmogorov-Smirnov test also identifies markers as significantly different that were less than 1 SD from average and identified markers as not different although the markers were 2 SD from the training set average.

To illustrate, in this experiment, 472 data points were analyzed, 415 were tested not different with both methods (less than 2 SD from average and identified not different with the Kolmogorov-Smirnov test), 41 were more than 1 SD from the average and considered different with the Kolmogorov-Smirnov test and for 9 data points the Kolmogorov-Smirnov method identified the data as different or not different (indicated with the black circles in FIG. 7), although the data points were less than 1 SD from average or more than 2 SD from average respectively. Comparing the dissociation enzyme-treated samples to the training set with the Kolmogorov-Smirnov test further improved the sensitivity and accuracy of the Kolmogorov-Smirnov test.

Comparison of Huvecs and epithelial cancer cell lines: Huvecs and several epithelial cancer cell lines were immunophenotyped and the immunophenotypes were compared. Table 7 shows the phenotypic differences when the cancer cell lines were compared to the Huvecs when a dMFI of 25 is considered significant. dMFIs between −10 and 10 are not shown. Table 8 shows markers that significantly differ between the BT-20, MDA-MB-231 and MCF-7 breast cancer cell lines. Table 9 shows the differences between PC-3 and DU-145 Prostate cancer cell lines.

In addition, the phenotypes were analyzed by origin of the cell lines.

Therefore the MFI values of the breast cancer cell lines were averaged and compared to the average of the two prostate cancer cell lines and compared to the M21 melanoma. The differences in expression are shown in Table 10.

Finally, the cell lines were qualitatively ranked in responsiveness to a compound, which may have anti-tumor properties. Huvecs and M21 are low responders to this compound, MCF-7 and PC-3 are intermediate responders and BT-20 and MDA-MB-231 are both high-responders. The response of DU-145 to the compound is not known. The MFI values were sorted in a low, intermediate and high-responsive group and analyzed for trends in MFI expression. Table 11 shows the results of analyzing the phenotypes grouped by responsiveness. The markers CD104, CD146, and CD58 show a trend in expression that could be correlated to their responsiveness to a compound, which may have anti-tumor properties. A cancer cell line with high CD104 expression, low CD146 expression and very high CD58 expression therefore is expected to be a high responder.

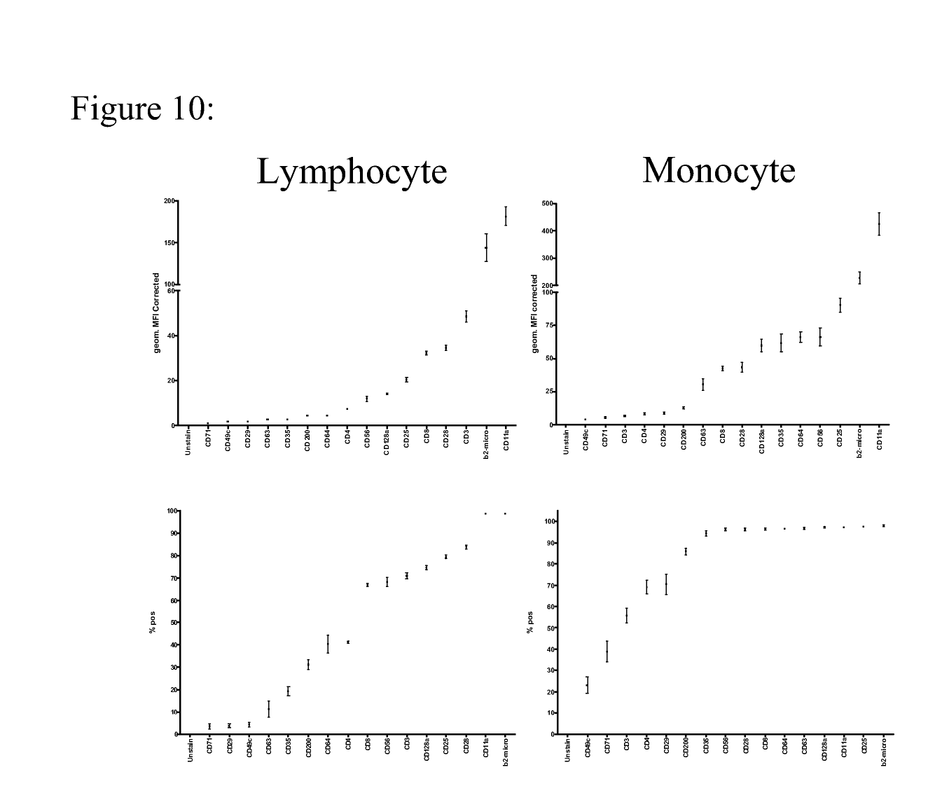

Heterogeneous cell populations: The next phase of research focused on immunophenotyping a heterogeneous cell population of PBMCs. Flow cytometry with whole blood is complicated because of the presence of erythrocytes. Therefore PBMCs must be isolated before analysis. The most conventional method for PBMC isolation is by gradient centrifugation, a method developed by Ficoll-Hypaque. Results of immunophenotyping PBMCs isolated with the Ficoll-Hypaque protocol are shown in FIG. 10 for the 17-marker panel.

A limitation of analyzing the percentage of positive cells is that it does not discriminate between intermediate and high expressed surface markers. When looking at the graph in FIG. 6, a marker with an MFI of 100 will be nearly 100% positive, but so will be a marker with a MFI of 1000, which is 10-fold upregulated. For visualization of the phenotypes therefore, the MFI-values are more useful than the percentage of positive cells.

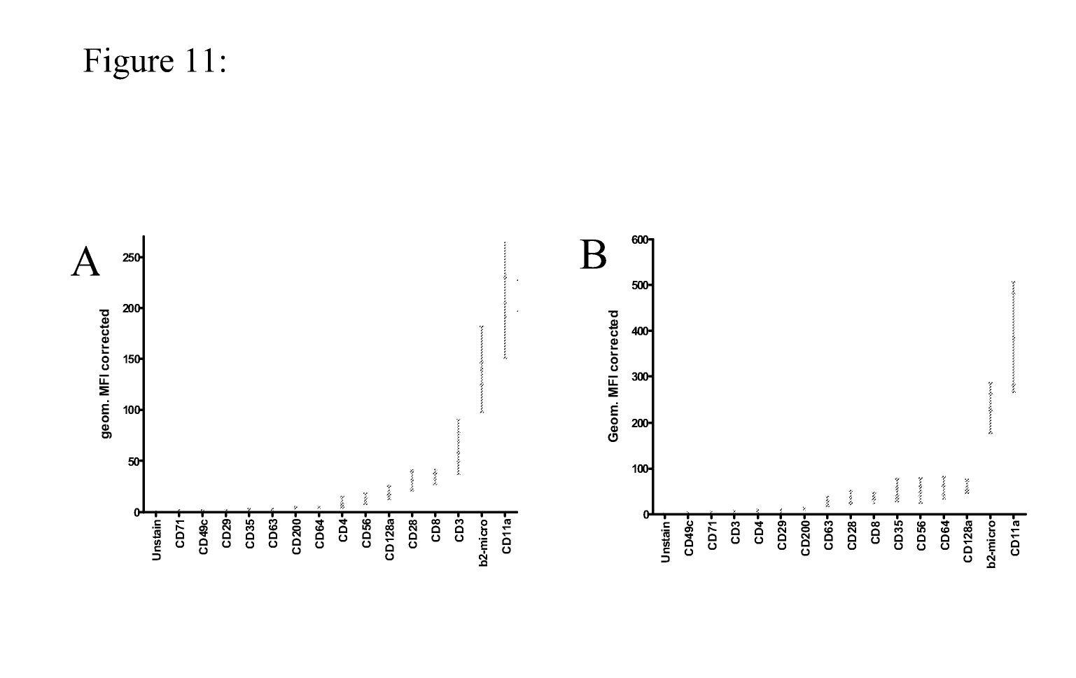

Comparison of methods: A limitation of the Ficoll-hypaque method is that it is time consuming and the protocol requires training of the experimenter and is less suitable for automation. Commercially ready-to-use blood collection tubes (CPT) based on the Ficoll-hypaque method are also available. The CPT isolated PBMCs were compared to conventional Ficoll-hypaque isolated PBMCs and the results are depicted in FIG. 11.

The differences in MFI between PBMCs isolated with both methods are very small. The variation between samples is also comparable with both methods. Because the cell preparation tubes are more easy to use and would be more suitable for automation than the conventional Ficoll-Hypaque method, isolation of PBMCs with the CPT tubes is preferred over Ficoll-Hypaque.

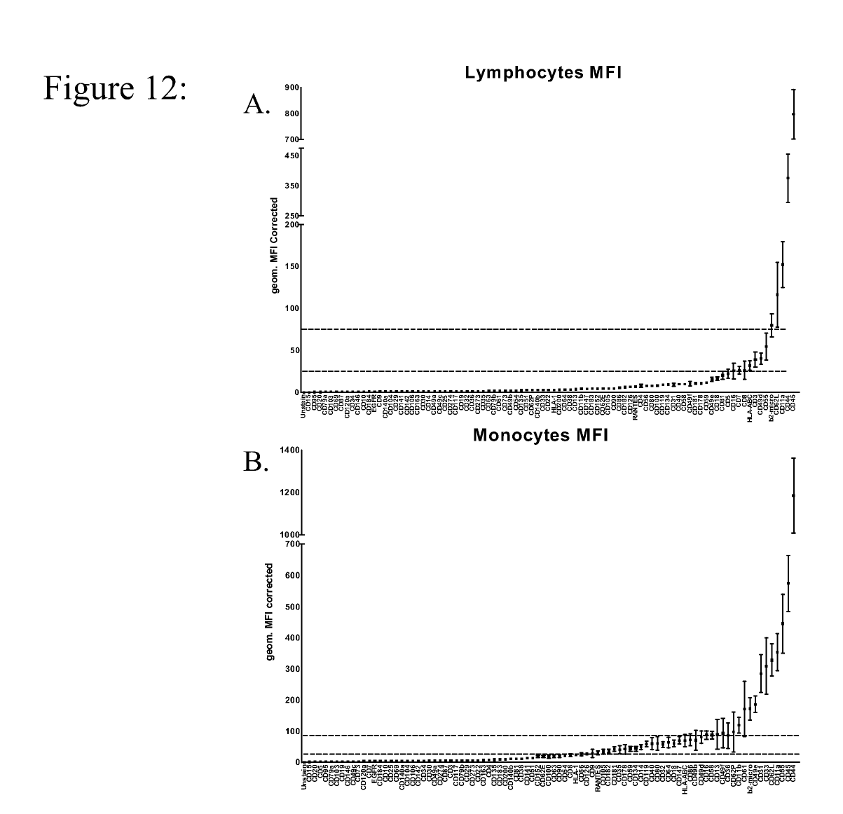

PBMC immunophenotype training set: To investigate the feasibility of immunophenotyping PBMCs for diagnostic, personalized medicine or predictive medicine purposes the immunophenotype of PBMCs of a group of healthy donors was analyzed. The results are shown in FIG. 12.

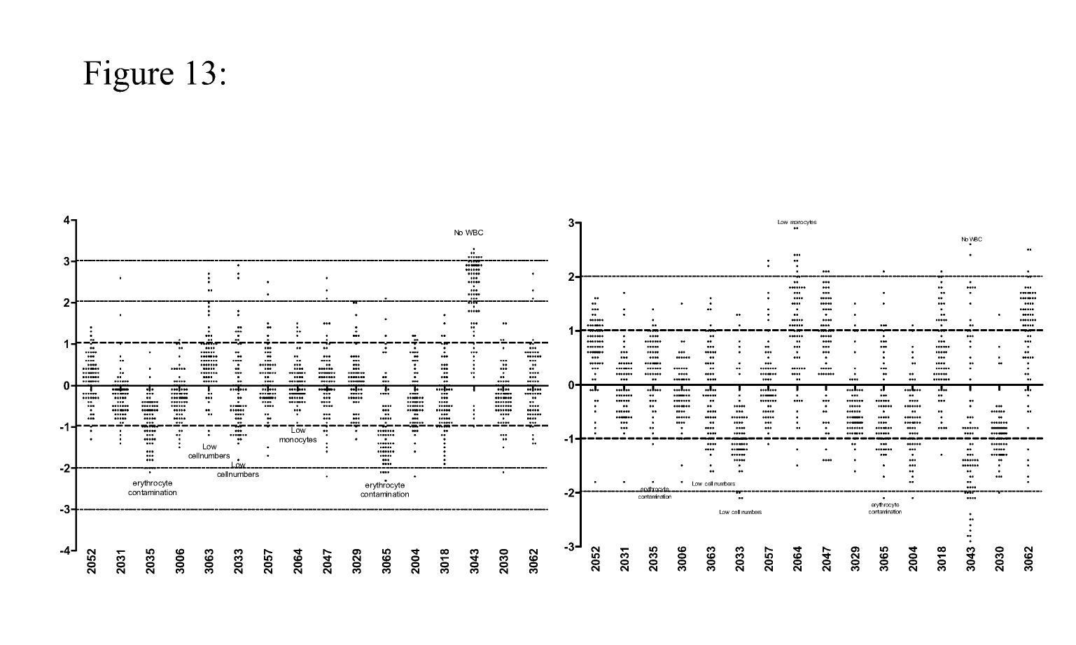

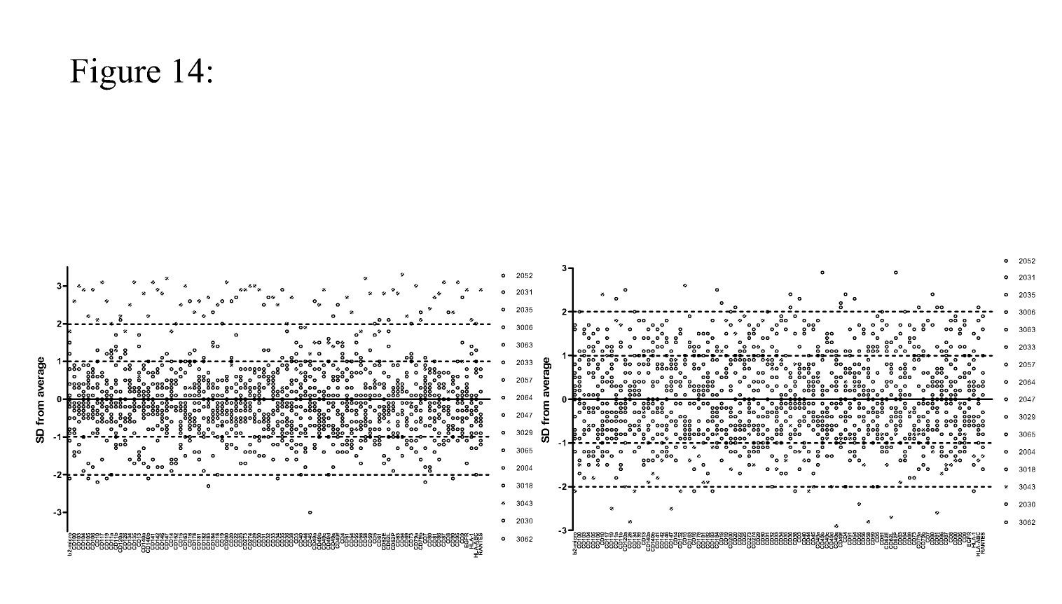

Some of these immunophenotyping experiments were performed under sub-optimal conditions due to technical errors and experimental errors. It is also important to test for unusual immune status of the donors. To distinguish what effect such errors have on the results, methods are needed to comment on the quality of the samples. Expressing the MFI values as standard deviations from the average (z=(xe−xav)/s) is a method to visualize the quality of the samples (see FIG. 13), where z is the number of standard deviations from the average, xe is the experimental MFI value, xav is the average MFI value and s is the standard deviation. Another possibility is to plot the same data (z=(xe−xav)/s) for each datapoint individually, which visualizes the variation of the marker between the different donors (see FIG. 14).

FIG. 13 visualizes that lymphocytes of the donors 2035 and 3065 have a majority of parameters that are one standard deviation below average, due to incomplete separation of erythrocytes and donors 2035 and 3065 were therefore excluded from the training set. Donors 2033, 3063, 2064 and 3043 all have an unusual standard deviation pattern and were also excluded from the training set. Samples that do not have the majority of MFIs between 1 standard deviation from the average may not meet quality requirements. The donors 2035 and 3065 had incomplete separation of erythrocytes resulting in a majority of lymphocyte markers with MFIs more than one standard deviation below average, monocytes however seem unaffected by the presence of erythrocytes. From the donors 2033 and 3063 low cell numbers were isolated due to experimental errors, which mostly affected the expression of monocytes. From donor 2064 low monocytes were isolated and from donor 3043 low cell numbers were isolated, indicating an altered immune status of these donors. Cell populations with a majority of markers that differ more than 1 standard deviations from the average were removed from the training set.

In FIG. 14 donor 3043 is colored red. From donor 3043 low cell white blood cell numbers were isolated, indicating an altered immune status. FIG. 14 shows that the altered immune status mostly increases the expression of surface markers were in monocytes the expression of surface markers is decreased. Such visualization methods can be used to exclude donors from a training set or to identify patients with altered immune status. For example, the donor 3043 is colored red. 3043 was a donor with altered immune status.

Discussion: The field of cytomics is a promising and emerging field of research, which adds up to the fields of genomics, and proteomics research. Flow cytometry provides one of the tools in the field of cytomics. The method for high throughput immunophenotyping, based on flow cytometry, shown in the present invention, is able to analyze the expression of many surface markers in a single test and it is a promising platform technology for developing a diagnostic test and to make personalized and predictive medicine a reality. The developed method for high throughput immunophenotyping can clearly be used to characterize both homogeneous as heterogeneous cell types.

To test the applicability of the immunophenotyping method three breast cancer (BT-20, MDA-MB-231 and MCF-7), two prostate cancer (DU-145 and PC-3) and one skin cancer cell line (M21) were analyzed for 90 parameters and compared with Huvecs. This experiment was not performed to research what the differences between the various cell lines actually mean. It was performed as proof of principle that the developed method can be used for high throughput immunophenotyping of cell lines.

The immunophenotype differs among more than twenty parameters, leaving about seventy markers unchanged. All of the parameters that are found to differ among the cell lines are related to tumor growth, progression and invasiveness. In table 12 the markers that differ significantly are summarized and an example of their relation to cancer is given. Many of these markers are differentially expressed among the cancer cell lines, which can be used to explain behavior of these cell lines. Explaining the behavior of cancer cell lines can be used to set prognosis or to predict the outcome of a therapy. Further research however is needed to validate these results by analyzing gene and protein expression of the markers that differ among the various cancer cell lines. Validation of the results will also enable selection and exclusion of false positive or false negative results. It is also needed to comment on the physiological meaning of these differences and to correlate the results to disease outcome or in vivo function. As mentioned before it was not a goal to investigate the phenotypes of several cancer cell lines, but the goal was to proof that our method is able to analyze the phenotype of homogeneous cell lines.

A method to analyze the phenotype of cell lines can be a valuable research tool. Besides characterizing cell lines it can also be used to monitor the effects of a drug. In addition, comparison of cell lines can be used to explain differences in responsiveness to a drug or to estimate whether a patient will be suffering from side effects.

For example, an antitumor drug has been tested on all cell lines except DU-145. The cell lines were subsequently ranked in their responsiveness to this drug. For three markers, CD104, CD146 and CD58 there was a trend in expression, which might be correlated to the effect of the drug. The drug has not been tested in the DU-145 prostate cancer cell line. DU-145 has a phenotype close to the intermediate responsive cell lines for the markers CD104, CD146 and CD58, suggesting that DU-145 might be an intermediate responder. It would be very interesting to test this hypothesis. Nevertheless, the developed method for immunophenotyping cells has been shown a very promising method to characterize homogeneous cell lines. There are however several challenging issues that require improvement and optimization for the heterogeneous cell types and especially PBMCs.

Different cell types require different methods of analysis. The percentage of positive cells for example cannot discriminate between cells that express a certain marker and cells that highly express a certain marker since in both situations near 100% of the cells is positive. Analysis with MFI values however will not be influenced much by small subpopulations of cells. Heterogeneous populations of cells, such as, for example, lymphocytes and monocytes, in contrast to homogeneous cell lines, often have a subpopulation of cells that is positive for certain markers were the main population of cells is not (for example activated lymphocytes), resulting in graphs. Other methods of analysis therefore are needed to analyze PBMCs.

As mentioned before, care should be taken to avoid bias of the experimenter in the analysis of flow cytometry data. Sources of variation are inconsistent gating strategies and using different machines and laboratories. To overcome these difficulties unbiased data analysis is needed. The Kolmogorov-Smirnov test is a promising method to analyze results in homogeneous cell populations as shown in FIGS. 8 and 9. Comparing the results of the Kolmogorov-Smirnov test with the results of FIG. 8 shows that the results of both methods show resemblance. The accuracy was further increased when the results were compared to a training set. However, further research is needed to fine-tune the Kolmogorov-Smirnov test.

To apply the Kolmogorov-Smirnov test to PBMC immunophenotyping data some modifications are needed. Because of incomplete separation of erythrocytes, debris and genetic variability, the scatters are inconsistent and the currently available Kolmogorov-Smirnov test cannot be applied.

There are two solutions to overcome these challenges. Firstly, different methods of isolating the PBMCs are available, resulting in samples with less erythrocytes and debris. The ficoll-hypaque based CPT-tube is an easy method to isolate monocytes and lymphocytes, which can be fully automated and does not involve much sample handling that can cause experimental variation. The results obtained with CPT-tube isolated PBMCs were comparable to those obtained with Fycoll-Hypaque, which is less easy to automate and requires more sample handling. A limitation of the CPT-tube is that it does not completely remove all erythrocytes and with varying effectiveness, which makes the Kolmogorov-Smirnov test inapplicable. Another popular method for isolation of PBMCs is lysis of erythrocytes. Lysing solutions are commercially available. An example of an erythrocyte lysing solution is BD FACS lysing solution. A limitation of BD FACS lysing solution is that it contains a fixative and that staining needs to be applied prior to lysis, which forces the use of a protocol to lyse each well individually. Other lysing solutions are also available, but the most of the lysis solutions contain fixatives and all lysing methods affect scatters and MFI values. For non-fixative lysis also ammonium chloride can be used. Ammonium chloride however also has been reported to affect MFI values. A limitation of all lysis methods therefore seems to be that they create artifacts and affect the measured MFI values. The second solution could be to make some adjustments to the Kolmogorov-Smirnov test to correct for erythrocytes and debris by writing an algorithm to gate out the erythrocytes and debris. A limitation of this method is that developing an algorithm will be time consuming and that the developer needs to set a gate, thereby reintroducing a bias.

If the method for immunophenotyping PBMCs will only be applied in a pattern recognition approach both of the above mentioned solutions are applicable. In pattern recognition it is not very important whether the measured expression is representative for the actual expression, as long as changes in expression (for example caused by disease) influence the measured expression significantly. The artifacts caused by lysis of erythrocytes will be reproducible and therefore will not affect the pattern. The artifacts however may exclude testing of certain markers, because the lysing solution diminishes expression of certain markers completely and changes in expression are lost. With the second solution, the adjustments to the Kolmogorov-Smirnov test will reintroduce the bias of the experimenter. It will however be the bias of only the developer of the initial algorithm and from there on all data will be analyzed with the same bias. This will mean that the events in the gate in the algorithm may not be completely representative for the actual cell population in the sample, but in pattern recognition sub optimal conditions are allowed as long as the results are reproducible. Probably, the most reliable data can be obtained by applying both proposed solutions. Improving erythrocyte separation increases accuracy and reproducibility although gates are used to exclude most of them. Including algorithm gates in the Kolmogorov-Smirnov test will also further reduce the effects of genetic and experimental variability.

Another complication of flow cytometry is that MFI values differ among flow cytometers and laboratories. To minimize the risk of misdiagnosis however, it is important to reduce experimental variation as much as possible. It might be possible to correct for variation in values by calibrating the system. The five wells that are reserved for set-up can also be used to adjust MFI values with the use of standard lyophilized cells, which are commercially available. Staining the lyophilized cells with an antibody with a known intensity against an antigen with a known and standardized expression level can be used to calibrate the flow cytometer. In the future, this calibration process can be fully automated.

Another cause of experimental variation is the concentration of the antibody staining. Transferring antibodies into a 96 wells-plate can cause variation. In addition, a decrease in intensity or affinity can occur over time. Lyophilized 96-wells plates filled with antibodies are commercially available from BD Biosciences. In these Lyoplates® minimal variation in antibody concentration will occur. In addition, every plate will contain antibodies with constant intensity and affinity.

To further improve the accuracy of diagnosis more quality control is needed. Donors with unusual marker expression due to experimental variation caused by experimental errors must be excluded from a training set and must be recognized among the patient population. An algorithm must be developed to automatically exclude such donors. Besides experimental errors MFI values have also been reported to be affected by sample preparation, temperature, anticoagulants and fixatives. Here we describe a method to visualize the quality of samples by expressing the MFI values as standard deviations (z=(xe−xav)/s) to create variations of the Sewhart chart. Expressing the values as standard deviations per sample clearly visualizes samples with incomplete separation of erythrocytes and without white blood cell layer in the lymphocyte chart (FIG. 13 left panel). The monocyte chart (FIG. 13 right panel) provides more variable distributions making it harder to interpret. It is also possible to express the same values individually per marker (FIG. 14). Using colors to highlight certain donors can be useful to select markers that are unusually expressed in some donors, where all other markers are normally expressed. Besides, an altered distribution of standard deviations could also be a sign of disease by itself. Until the Kolmogorov-Smirnov test is applicable this method of visualization can be used to screen for differences in expression in the patient population. It is also possible to use color identification to automate the process of quality control.

There are many potential applications of this method for immunophenotyping heterogeneous cell populations. For homogeneous cell lines fewer applications are possible. Isolating homogeneous cell population from patients is often invasive and in case of tumors biopsies are not without any health risks. For in vitro research purposes the immunophenotyping method could be a valuable addition. As shown with the cancer cell lines the sensitivity of the method shows potential to identify new drug targets and biomarkers. In the experiment in which dissociation enzymes were compared, differences in expression caused by treatment were measured with the immunophenotyping method, creating a system response profile (SRP). For research purposes a method to test for SRPs is highly valuable. It can be used to monitor effectiveness of therapy. In addition, analyzing SRPs can be used to unreveal working mechanisms and to increase our understanding of systems biology. For in vitro research the concept of pattern recognition is less appropriate and validation of the results becomes more important.

For heterogeneous cell populations and especially for PBMCs there are more applications. PBMCs are in contact with many cell types and have been shown to interact with disease-affected cells. It is hypothesized that all diseases will have an effect on the phenotype of PBMCs, thereby altering the SRP of PBMCs. To test this hypothesis, methods for multiparameter phenotyping are needed, because it is hard to predict which markers will be most likely to change. An unbiased multiparametric approach will have more chance of revealing markers that are subject to change in various disease models. In addition, a multiparametric approach will also increase the specificity of the results. For example, it is very likely that several diseases will all affect a marker, it is however less likely that several diseases all affect the same five markers. For the purpose of diagnosis revealing several markers that are changed in disease increases the certainty of diagnosis compared to one marker, which increases chances of accurate diagnosis. Besides diagnosis the method for immunophenotyping can also be used to compare the SRPs of treated patients, which can be used to monitor treatment.

The developed method for high throughput immunophenotyping may also be applicable in the field of predictive medicine. Genetic variability causes differential expression patterns, which cause variation in effectiveness of therapy and drug side-affects. Methods to identify patient subpopulations that are non-responders or that will suffer from side-effects can be used to enroll responsive patients in clinical trials and exclude non-responders, thereby streamlining the approval process of new drugs entering clinical trials. In addition, characterizing the phenotypes of PBMCs can also be used to select which therapy is most likely to succeed in a patient, allowing for more effective and more personalized medicine.

These possible applications of the method for immunophenotyping may not be realized in the near future. The possible applications of immunophenotyping PBMCs are based on theories and hypotheses that will require intensive research to test them. Here it is demonstrated that it is possible to characterize PBMCs. In order for this method to be a success it is required to confirm that PBMCs are subject to change in disease models and that the method for immunophenotyping is able to identify these changes. Several examples are yet available. For example, it has been reported that Mac-1 and LFA-1 (CD11a, CD11b and CD18) expression on leukocytes can be used to predict restenosis. It is possible that diseases will only affect a small subpopulation of PBMCs, which cannot be identified without further improving the sensitivity of the immunophenotyping method. The current method allows testing of 90 markers per test. It is very likely that markers that change in diseased subjects are not in the selection of 90 markers. Increasing the number of parameters per test is therefore of great importance. Increasing the number of parameters will also increase the costs. Since for most diseases it is unknown how they affect PBMCs it is also important to include intracellular signaling markers, cytokines and other immunogetic molecules.

Currently, the number of healthy and diseased subjects needed to be able to statistically identify disease markers for diagnosis of personalized and predictive medicine cannot be calculated. Due to genetic variability it is possible that thousands of healthy donors are needed to establish a non-disease SRP and also hundreds of patients are needed to analyze the SRP of a disease. It has also been suggested that expression of several surface markers is affected by age, which would further increase the number of patients required to assure statistical relevance.

There are many possible diseases in which improved methods for diagnosis are needed. For the success of the method for immunophenotyping PBMCs it is however important to start with a disease that is known to affect leukocytes for proof of concept. Such a common disease, which lacks methods for diagnosis in the early onset of the disease, is diabetes type II. Diabetes type II has been reported to affect the expression of several surface markers, such as CD11b, CD14, CD18, the LDL receptor and CD36 on monocytes. Using the method for immunophenotyping PBMCs for predictive and personalized medicine will require much more research and therefore the chance of success in the near future is currently less than the chance of success in diagnostics.

Conclusion: In summary, the developed method for high throughput immunotherapy is able to analyze expression of surface markers in a single test in both homogeneous and heterogeneous cell types and is a promising technique for diagnosis of many diseases and as a basic research tool. The potential of analyzing homogeneous cell types has been illustrated by characterizing and comparing several cancer cell lines and has potential usage as a basic research tool. Analysis of heterogeneous cell types and especially PBMCs could become a valuable diagnostic tool, but there are many challenging issues that need to be solved. The Kolmogorov-Smirnov test has potential as a test to analyze immunophenotypic patterns. However, the test needs more fine-tuning. For the purpose of diagnosis, quality control also is of great importance. Here we describe variations of the Sewhart chart to visualize the quality of the samples.

From these data it can be concluded that high throughput immunophenotyping has potential as a basic research tool and as a diagnostic test. Both applications however still require to be further developed.

Future prospects: For applications as a basic research tool validation of the results is needed. Therefore the results of phenotyping the various cancer cell lines should be verified by analyzing gene expression and protein expression of the surface markers that differ among the various cell lines. Furthermore, it would be interesting to correlate protein expression to in vivo behavior of the cancer cell lines.

It is also of great importance to make this test applicable for phenotyping PBMCs. It is therefore a first priority to further evaluate methods to improve isolation of PBMCs. Although non-fixating erythrocyte lysing solutions create artifacts, they might reduce experimental variation and improve removal of erythrocytes. Lysis of erythrocytes could therefore become the protocol of choice. In addition, it is also a priority to improve the Kolmogorov-Smirnov test by writing an algorithm that excludes erythrocytes and debris to further reduce the effects of experimental variation.

To develop a diagnostic test of the immunophenotyping method more research is needed to test the hypothesis that diseases affect leukocyte expression levels in such a way that it can be measured by flow cytometry in a pattern recognition approach. It is therefore recommended to test this hypothesis for surface markers that are known to change in a certain disease first and not search for new disease biomarkers until the method for immunophenotyping is optimized and validated, which increases the chance of success.

Other challenging issues are in reducing experimental variation. Using standardized stained cells can be used to correct for variation between machines and labs. Using standardized Lyophilized plates can be used to further reduce experimental variation.

TABLE 1
PHENOTYPIC MARKERS SUITABLE FOR USE IN THE PRESENT
INVENTION

| CD identification | MOLECULE | Gene Name | Fluorescence | company |
|---|---|---|---|---|
| CD3 | CD3/T-cell antigen | CD3G/Z | PE | Caltag |
| | receptor (TCR) | | | |
| CD4 | OKT4, Leu 3a, T4 | CD4 | PE | BD Pharmingen |
| CD5 | T1, Leu1 | CD5 | PE | BD PharMingen |
| CD7 | Leu 9, 3A1, gp40, T | CD7 | PE | BD Pharmingen |
| | cell leukemia | | | |
| | antigen | | | |
| CD8 | MHC class I | CD8 | PE | BD PharMingen |
| | receptor | | | |
| CD9 | type III | CD9 | PE | BD PharMingen |
| | transmembrane | | | |
| | protein (platelet | | | |
| | aggregation/activation) | | | |
| CD10 | CALLA, membrane | MME | PE | BD Pharmingen |
| | metallo- | | | |
| | endopeptidase | | | |
| CD11a | alphaL; LFA-1, | ITGAL | PE | BD Pharmingen |
| | gp180/95 | | | |
| CD13 | Aminopeptidase N, | ANPEP | PE | BD Pharmingen |
| | APN, gp150, EC | | | |
| | 3.4.11.2 | | | |
| CD15 | fucosyltransferase 4 | FUT4 | PE | BD PharMingen |
| | (alpha (1,3) | | | |
| | fucosyltransferase, | | | |
| | myeloid-specific) | | | |
| CD16 | Fc gamma R III | FCGR3A | PE | Caltag |
| CD16a | Fc gamma R IIIa | FCGR3A | PE | BD Pharmingen |
| CD18 | β2-Integrin chain, | ITGB2 | PE | BD Pharmingen |
| | macrophage antigen | | | |
| | 1 (mac-1) | | | |
| CD19 | B4, associated with | CD19 | PE | BD PharMingen |
| | CD21, CD81, | | | |
| | CD225, Leu-13, | | | |
| | Lyn, Fyn, Vav, PI3- | | | |
| | kinase | | | |
| CD20 | membrane-spanning | MS4A1 | PE | BD PharMingen |
| | 4-domains, | | | |
| | subfamily A, | | | |
| | member 1 | | | |
| CD21 | C3d receptor, CR2, | CR2 | PE | BD Pharmingen |
| | gp140; EBV | | | |
| | receptor | | | |
| CD22 | Bgp135; BL-CAM, | CD22 | FITC | BD Pharmingen |
| | Siglec2 | | | |
| CD25 | Interleukin-2 | IL2RA | PE | BD PharMingen |
| | receptor (IL-2R, | | | |
| | inflammatory | | | |
| | response) | | | |
| CD29 | Integrin β1 chain; | ITGB1 | PE | BD Pharmingen |
| | platelet GPIIa; VLA | | | |
| | (CD49) beta-chain | | | |
| CD30 | tumor necrosis | TNFRSF8 | PE | BD PharMingen |
| | factor receptor | | | |
| | superfamily, | | | |
| | member 8. KI-1 | | | |
| CD31 | PECAM-1; platelet | PECAM1 | PE | BD Pharmingen |
| | GPIIa′; endocam | | | |
| CD32 | Fcgamma receptor | FCGR2A | PE | BD Pharmingen |
| | type II (FcgRII), | | | |
| | gp40 | | | |
| CD33 | gp67 | CD33 | PE | BD PharMingen |
| CD34 | My10, gp105-120 | CD34 | PE | BD PharMingen |
| CD35 | C3b/C4b receptor; | CR1 | PE | BD Pharmingen |
| | complement receptor | | | |
| | type 1 (CR1) | | | |
| CD36 | platelet GPIV, | CD36 | PE | BD Pharmingen |
| | GPIIIb, OKM-5 | | | |
| | antigen | | | |
| CD38 | T10; gp45, ADP- | CD38 | PE | BD Pharmingen |
| | ribosyl cyclase | | | |
| CD40 | Bp50, TNF Receptor 5 | TNFRSF5 | PE | BD PharMingen |
| CD44 | Pgp-1; gp80-95, | CD44 | PE | BD PharMingen |
| | Hermes antigen, | | | |
| | ECMR-III and | | | |
| | HUTCH-I. | | | |
| CD45 | LCA, B220, protein | PTPRC | PE | BD Pharmingen |
| | tyrosine | | | |
| | phosphatase, | | | |
| | receptor type, C | | | |
| CD49a | Integrin a1 chain, | ITGA1 | PE | BD Pharmingen |
| | very late antigen, | | | |
| | VLA 1a | | | |
| CD49b | Integrin a2 chain, | ITGA2 | PE | BD Pharmingen |
| | VLA-2-alpha chain, | | | |
| | platelet gpla | | | |
| CD49c | Integrin a3 chain, | ITGA3 | PE | BD Pharmingen |
| | VLA-3 alpha chain | | | |
| CD49d | Integrin a4 chain,, | ITGA4 | PE | BD Pharmingen |
| | VLA-4-alpha chain | | | |
| CD49e | Integrin a5 chain,, | ITGA5 | PE | BD Pharmingen |
| | VLA-5 alpha chain | | | |
| CD49f | Integrin a6 chain,, | ITGA6 | PE | BD Pharmingen |
| | VLA-6 alpha chain, | | | |
| | platelet gpIc | | | |
| CD50 | ICAM-3, | ICAM3 | fitc | BD Pharmingen |
| | intercellular | | | |
| | adhesion molecule 3 | | | |
| CD51 | Integrin alpha chain, | ITGAV | PE | BD Pharmingen |
| | vitronectin receptor | | | |
| | a chain | | | |
| CD54 | ICAM-1, | ICAM1 | PE | BD Pharmingen |
| | intercellular | | | |
| | adhesion molecule 1 | | | |
| CD55 | decay-accelerating | DAF | PE | BD PharMingen |
| | factor (DAF, prevent | | | |
| | cell damage) | | | |
| CD56 | Neural cell adhesion | NCAM1 | PE | Caltag |
| | molecule 1 | | | |
| | (NCAM1), NKH-1 | | | |
| | (NK cell marker) | | | |
| CD58 | Lymphocyte | CD58 | PE | BD Pharmingen |
| | function-associated | | | |
| | antigen-3 (LFA-3, | | | |
| | interacts with CD2 | | | |
| | during cell adhesion) | | | |
| CD59 | MACIF, MIRL, P- | CD59 | PE | BD Pharmingen |
| | 18, protectin | | | |
| CD61 | Glycoprotein IIIa, | ITGB3 | PE | BD Pharmingen |
| | beta3 integrin | | | |
| CD62E | E-selectin, LECAM- | SELE | PE | BD Pharmingen |
| | 2, ELAM-1 | | | |
| CD62L | L-selectin, LAM-1, | SELL | PE | Caltag |
| | Mel-14 | | | |
| CD62P | P-selectin, granule | SELP | PE | BD Pharmingen |
| | membrane protein- | | | |
| | 140 (GMP-140) | | | |
| CD63 | LIMP, gp55, | CD63 | PE | BD Pharmingen |
| | LAMP-3 | | | |
| | neuroglandular | | | |
| | antigen, | | | |
| | granulophysin | | | |
| CD64 | FcgR1, FcgammaR1 | FCGR1A | PE | Caltag |
| | (IgG-receptor) | | | |
| CD69 | Early T-cell | CD69 | PE | BD PharMingen |
| | activation antigen | | | |
| CD71 | Transferrin receptor | TFRC | PE | BD Pharmingen |
| CD73 | Ecto-5′-nucleotidase | NT5E | PE | BD Pharmingen |
| CD79a | CD79A antigen | CD79A | PE | BD PharMingen |
| | (immunoglobulin- | | | |
| | associated alpha), | | | |
| | MB1 | | | |
| CD79b | CD79B antigen | CD79B | PE | BD PharMingen |
| | (immunoglobulin- | | | |
| | associated beta), | | | |
| | B29 | | | |
| CD80 | B7-1; BB1 | CD80 | PE | BD Pharmingen |
| CD81 | Target of an | CD81 | PE | BD Pharmingen |
| | antiproliferative | | | |
| | antibody (TAPA-1); | | | |
| | M38 | | | |
| CD83 | HB15 | CD83 | PE | BD Pharmingen |
| CD86 | B7-2; B70 | CD86 | PE | Caltag |
| | | | | Laboratories |
| CD87 | plasminogen | PLAUR | PE | BD PharMingen |
| | activator, urokinase | | | |
| | receptor (PLAUR), | | | |
| | uPAR | | | |
| CD88 | C5a-receptor | C5R1 | PE | BD Pharmingen |
| CD90 | Thy-1 | THY1 | PE | BD PharMingen |
| CD95 | APO-1, Fas, | TNFRSF6 | PE | BD Pharmingen |
| | TNFRSF6 | | | |
| CD100 | SEMA4D | SEMA4D | PE | Serotec |
| CD103 | integrin, alpha E | ITGAE | PE | BD PharMingen |
| | (human mucosal | | | |
| | lymphocyte antigen | | | |
| | 1; alpha | | | |
| | polypeptide) | | | |
| CD104 | Integrin beta 4 | ITGB4 | PE | BD Pharmingen |
| | subunit, TSP-1180 | | | |
| CD105 | Endoglin | ENG | PE | Caltag |
| CD106 | VCAM-1 (vascular | VCAM1 | PE | BD Pharmingen |
| | cell adhesion | | | |
| | molecule-1), | | | |
| | INCAM-110 | | | |
| CD114 | G-CSFR, HG-CSFR, | CSF3R | PE | BD Pharmingen |
| | CSFR3 | | | |
| CD117 | SCFR, c-kit, stem | KIT | PE | BD Biosciences |
| | cell factor receptor | | | |
| CD119 | IFN gamma receptor | IFNGR1 | PE | BD Pharmingen |
| | alpha chain (GIR- | | | |
| | 208) | | | |
| CD120b | TNFRI; TNFRp55 | TNFRSF1B | PE | BD Pharmingen |
| CD126 | IL-6 receptor alpha | IL6R | PE | BD Pharmingen |
| | chain | | | |
| CD134 | Tumor Necrosis | TNFRSF4 | PE | Caltag |
| | Factor receptor 4, | | | |
| | OX40 (promotes | | | |
| | expression of | | | |
| | BCL2/BCL-xL) | | | |
| CD135 | fms-related tyrosine | FLT3 | PE | BD PharMingen |
| | kinase 3 | | | |
| CD140b | b-platelet derived | PDGFRB | PE | BD Pharmingen |
| | growth factor | | | |
| | (PDGF) receptor | | | |
| CD141 | Thrombomodulin | THBD | PE | BD Pharmingen |
| | (TM), fetomodulin | | | |
| CD142 | Tissue factor, | F3 | PE | BD Pharmingen |
| | thromboplastin, | | | |
| | coagulation factor | | | |
| | III | | | |
| CD146 | Muc 18, MCAM, | MCAM | PE | BD Pharmingen |
| | Mel-CAM, s-endo | | | |
| CD147 | Basigin, M6, | BSG | PE | Serotec |
| | extracellular | | | |
| | metalloproteinase | | | |
| | inducer | | | |
| | (EMMPRIN) | | | |
| CD151 | Platelet-endothelial | CD151 | PE | BD Pharmingen |
| | tetra-span antigen | | | |
| | (PETA)-3 | | | |
| CD152 | Cytolytic T | CTLA4 | PE | BD PharMingen |
| | lymphocyte- | | | |
| | associated antigen | | | |
| | (CTLA-4) | | | |
| CD163 | M130, GHI/61, | CD163 | PE | BD PharMingen |
| | RM3/1 | | | |
| CD164 | MUC-24, MGC 24, | CD164 | PE | BD Pharmingen |
| | multi-glycosylated | | | |
| | core protein 24 | | | |
| CD165 | AD2, gp 37 | | PE | BD Pharmingen |
| CD178 | FAS ligand, CD95 | TNFSF6 | PE | Caltag |
| | ligand | | | Laboratories |
| CD181 | CXCR1 CDw128a: | IL8RA | PE | BD Pharmingen |
| | IL-8 receptor alpha, | | | |
| CD182 | CXCR2 | IL8RB | PE | BD Pharmingen |
| CD183 | CXCR3 chemokine | CXCR3 | PE | BD Pharmingen |
| | receptor, G protein- | | | |
| | coupled receptor 9 | | | |
| CD184 | CXCR4 chemokine | CXCR4 | PE | BD Pharmingen |
| | receptor, Fusin | | | |
| CD200 | OX2 (regulates Mf | CD200 | PE | BD PharMingen |
| | activity) | | | |
| CD273 | B7DC, PDL2 | PDCD1LG2 | PE | BD Pharmingen |
| CD274 | B7H1, PDL1 | PDCD1LG1 | PE | BD Pharmingen |
| CD275 | B7H2, ICOSL | ICOSL | | |
| CD276 | B7H3 | N/A | | |
| CD277 | BT3.1 | BTN3A1 | | |
| CD278 | ICOS | ICOS | | |
| CD279 | PD1 | PDCD1 | | |
| CD280 | ENDO180 | MRC2 | | |
| CD281 | TLR1 | TLR1 | | |
| CD282 | TLR2 | TLR2 | | |
| CD283 | TLR3 | TLR3 | | |
| CD284 | TLR4 | TLR4 | | |
| CD289 | TLR9 | TLR9 | | |
| CD292 | BMPR1A | BMPR1A | | |
| CDw293 | BMPR1B | BMPR1B | | |
| CD294 | CRTH2 | GPR44 | | |
| CD295 | LeptinR | LEPR | | |
| CD296 | ART1 | ART1 | | |
| CD297 | ART4 | DO | | |
| CD298 | Na+/K+-ATPase b3 | ATP1B3 | | |
| CD299 | DCSIGN-related | CD209L | | |
| CD300a | CMRF35H | | | |
| CD300c | CMRF35A | | | |
| CD300e | CMRF35L1 | | | |
| CD301 | MGL, CLECSF14 | CLECSF14 | | |
| CD302 | DCL1 | N/A | | |
| CD303 | BDCA2 | CLECSF7 | | |
| CD304 | BDCA4, Neuropilin 1 | NRP1 | | |
| CD305 | LAIR1 | LAIR1 | | |
| CD306 | LAIR2 | LAIR2 | | |
| CD307 | IRTA2 | N/A | | |
| CD309 | VEGFR2, KDR | KDR | | |
| CD312 | EMR2 | EMR2 | | |
| CD314 | NKG2D | KLRK1 | | |
| CD315 | CD9P1 | PTGFRN | | |
| CD316 | EWI2 | IGSF8 | | |
| CD317 | BST2 | BST2 | | |
| CD318 | CDCP1 | N/A | | |
| CD319 | CRACC | SLAMF7 | | |
| CD320 | 8D6A | N/A | | |
| CD321 | JAM1 | F11R | | |
| CD322 | JAM2 | JAM2 | | |
| CD324 | E-Cadherin | CDH1 | | |
| CDw325 | N-Cadherin | CDH2 | | |
| CD326 | Ep-CAM | TACSTD1 | | |
| CDw327 | siglec6 | SIGLEC6 | | |
| CDw328 | siglec7 | SIGLEC7 | | |
| CDw329 | siglec9 | SIGLEC9 | | |
| CD331 | FGFR1 | FGFR1 | | |
| CD332 | FGFR2 | FGFR2 | | |
| CD333 | FGFR3 | FGFR3 | | |
| CD334 | FGFR4 | FGFR4 | | |
| CD335 | NKp46 | NCR1 | | |
| CD336 | NKp44 | NCR2 | | |
| CD337 | NKp30 | NCR3 | | |
| CDw338 | ABCG2, BCRP | ABCG2 | | |
| CD339 | Jagged-1 | JAG1 | | |
| ABCG2 | | | | |
| alkaline | | | | |
| phosphatase | | | | |
| BCL-xL | BCL2 like-1 | BCL2L1 | PE | Chemicon |
| bCL-2 | B-cell | BCL2 | PE | BD Pharmingen |
| | CLL/lymphoma-2 | | | |
| CK 5 | Cytokeratin 5 | | | |
| CK 6 | Cytokeratin 6 | | | |
| CK 7 | Cytokeratin 7 | | | |
| CK 8 | Cytokeratin 8 | | | |
| CK 10 | Cytokeratin 10 | | | |
| CK 13 | Cytokeratin 13 | | | |
| CK 14 | Cytokeratin 14 | | | |
| CK 15 | Cytokeratin 15 | | | |
| CK 16 | Cytokeratin 16 | | | |
| CK 18 | Cytokeratin 18 | | | |
| CK 19 | Cytokeratin 19 | | | |
| EGFr | Epidermal growth | EGFR | PE | BD PharMingen |
| | factor receptor | | | |
| | (growth and | | | |
| | differentiation) | | | |
| HGF-R | Hepatocyte growth | HGFR | | |
| | factor receptor | | | |
| NGFr | NGFR (p75) | NGFR | PE | BD Pharmingen |
| RANTES | CCL5 (chemokine | CCL5 | PE | Caltag |
| | (C-C motif) ligand 5 | | | |
| b2-microglobulin | Associates with | B2M | PE | BD Pharmingen |
| | HLA class I antigen | | | |
| | complex | | | |
| HLA-ABC | MHC Class I ABC | | PE | BD Pharmingen |
| HLA-DP | MHC II HLA-DP | HLA-DPB1 | PE | Serotec |
| | beta-1 | | | |
| HLA DR | | | PE | BD Pharmingen |
| SSEA-1 | Embryonic Stem | SSEA1 | | |
| | Cell Antigen 1 | | | |
| SSEA-3 | Embryonic Stem | SSEA3 | | |
| | Cell Antigen 3 | | | |
| SSEA-4 | Embryonic Stem | SSEA4 | | |
| | Cell Antigen 4 | | | |

TABLE 2
AN EXAMPLE OF A STEM CELL PHENOTYPIC MARKER ARRAY PLATE

| | 1 | 2 | 3 | 4 | 5 | 6 | 7 | 8 | 9 | 10 | 11 | 12 |
|---|---|---|---|---|---|---|---|---|---|---|---|---|
| A | Unstain | Unstain | Unstain | Unstain | Unstain | Unstain | CD9 | CD10 | CD11a | CD13 | CD14 | CD16 |
| B | CD18 | CD25 | CD29 | CD31 | CD32 | CD34 | CD35 | CD36 | CD40 | CD44 | CD45 | CD49a |
| C | CD49b | CD49c | CD49d | CD49e | CD49f | CD51 | CD54 | CD55 | CD58 | CD59 | CD61 | CD62E |
| D | CD62L | CD62P | CD63 | CD64 | CD71 | CD73 | CD80 | CD81 | CD86 | CD90 | CD95 | CD100 |
| E | CD104 | CD105 | CD106 | CD117 | CD119 | CD120a | CD126 | CD134 | CD140a | CD140b | CD141 | CD142 |
| F | CD146 | CD147 | CD178 | CD181 | CD182 | CD183 | CD184 | CD200 | CD273 | CD274 | □2- | HLA- |
| | | | | | | | | | | | microglobulin | ABC |
| G | HLA- | EGFR | NGFR | RANTES | CD3 | CD4 | CD7 | CD15 | CD19 | CD20 | CD21 | CD22 |
| | DP | | | | | | | | | | | |
| H | CD38 | CD50 | CD62L | CD83 | CD88 | CD112 | CD114 | CD151 | CD164 | CD165 | CD172 | HLA- |
| | | | | | | | | | | | | DR |

TABLE 3
AN EXAMPLE OF AN ONCOLOGY PHENOTYPIC MARKER ARRAY PLATE

| | 1 | 2 | 3 | 4 | 5 | 6 | 7 | 8 | 9 | 10 | 11 | 12 |
|---|---|---|---|---|---|---|---|---|---|---|---|---|
| A | Unstain | Unstain | Unstain | Unstain | Unstain | Unstain | CD3 | CD4 | CD5 | CD7 | CD8 | CD9 |
| B | CD10 | CD11a | CD13 | CD14 | CD15 | CD16 | CD18 | CD19 | CD20 | CD22 | CD25 | CD29 |
| C | CD30 | CD31 | CD32 | CD33 | CD34 | CD35 | CD36 | CD38 | CD40 | CD44 | CD45 | CD49a |
| D | CD49b | CD49c | CD49d | CD49e | CD49f | CD51 | CD54 | CD55 | CD56 | CD58 | CD59 | CD61 |
| E | CD62E | CD62L | CD62P | CD63 | CD64 | CD69 | CD71 | CD73 | CD79a | CD79b | CD80 | CD81 |
| F | CD86 | CD87 | CD90 | CD95 | CD100 | CD103 | CD104 | CD105 | CD106 | CD117 | CD119 | CD120a |
| G | CD126 | CD134 | CD135 | CD140a | CD140b | CD141 | CD142 | CD146 | CD147 | CD152 | CD163 | CD178 |
| H | CD181 | CD182 | CD183 | CD184 | CD200 | CD273 | CD274 | b2- | EGFR | HLA- | HLA- | RANTES |
| | | | | | | | | micorglobulin | | ABC | 1 (DP) | |

TABLE 4-A
CELL SURFACE MARKERS USED FOR IMMUNOPHENOTYPING

| | | Gene | |
|---|---|---|---|
| | MOLECULE | Name | Source |
| CD3 | CD3/T-cell antigen receptor (TCR) | CD3G/Z | Caltag |
| CD4 | MHC class II receptor | CD4 | BD |
| | | | PharMingen |
| CD5 | T1, Leu1 | CD5 | BD |
| | | | PharMingen |
| CD7 | p41, GP40 | CD7 | BD |
| | | | PharMingen |
| CD8 | MHC class I receptor | CD8 | BD |
| | | | PharMingen |
| CD9 | Type III transmembrane protein (platelet | CD9 | BD |
| | aggregation/activation) | | PharMingen |
| CD10 | CALLA, membrane metallo-endopeptidase | MME | BD |
| | | | Pharmingen |
| CD11a | alphaL; LFA-1, gp180/95 | ITGAL | BD |
| | | | Pharmingen |
| CD13 | Amino peptidase N, APN, gp150, EC 3.4.11.2 | ANPEP | BD |
| | | | Pharmingen |
| CD14 | Surface receptor for LPS and serum LPS-binding | CD14 | BD |
| | protein (LBP) | | PharMingen |
| CD15 | Fucosyltransferase 4 (alpha (1,3) fucosyltransferase, | FUT4 | BD |
| | myeloid-specific) | | PharMingen |
| CD16a | Fc gamma R IIIa | FCGR3A | BD |
| | | | Pharmingen |
| CD18 | β2-Integrin chain, macrophage antigen 1 (mac-1) | ITGB2 | BD |
| | | | Pharmingen |
| CD19 | B4, associated with CD21, CD81, CD225, Leu-13, | CD19 | BD |
| | Lyn, Fyn, Vav, PI3-kinase | | PharMingen |
| CD20 | Membrane-spanning 4-domains, subfamily A, | MS4A1 | BD |
| | member 1 | | PharMingen |
| CD22 | BL-CAM | CD22 | Caltag |
| CD25 | Interleukin-2 receptor (IL-2R, inflammatory | IL2RA | BD |
| | response) | | PharMingen |
| CD29 | Integrin β1 chain; platelet GPIIa; VLA (CD49) beta- | ITGB1 | BD |
| | chain | | Pharmingen |
| CD30 | Tumor necrosis factor receptor super family, | TNFRSF8 | BD |
| | member 8. KI-1 | | PharMingen |
| CD31 | PECAM-1; platelet GPIIa′; endocam | PECAM1 | BD |
| | | | Pharmingen |
| CD32 | Fcgamma receptor type II (FcgRII), gp40 | FCGR2A | BD |
| | | | Pharmingen |
| CD33 | gp67 | CD33 | BD |
| | | | PharMingen |
| CD34 | My10, gp105-120 | CD34 | BD |
| | | | PharMingen |
| CD35 | C3b/C4b receptor; complement receptor type 1 | CR1 | BD |
| | (CR1) | | Pharmingen |
| CD36 | Platelet GPIV, GPIIIb, OKM-5 antigen | CD36 | BD |
| | | | Pharmingen |
| CD38 | T10 (cell adhesion, signal transduction) | CD38 | BD |
| | | | PharMingen |
| CD40 | Bp50, TNF Receptor 5 | TNFRSF5 | BD |
| | | | PharMingen |
| CD44 | Pgp-1; gp80-95, Hermes antigen, ECMR-III and | CD44 | BD |
| | HUTCH-I. | | PharMingen |
| CD45 | LCA, B220, protein tyrosine phosphatase, receptor | PTPRC | BD |
| | type, C | | Pharmingen |
| CD49a | Integrin a1 chain, very late antigen, VLA 1a | ITGA1 | BD |
| | | | Pharmingen |
| CD49b | Integrin a2 chain, VLA-2-alpha chain, platelet gpla | ITGA2 | BD |
| | | | Pharmingen |
| CD49c | Integrin a3 chain, VLA-3 alpha chain | ITGA3 | BD |
| | | | Pharmingen |
| CD49d | Integrin a4 chain, VLA-4-alpha chain | ITGA4 | BD |
| | | | Pharmingen |
| CD49e | Integrin a5 chain, VLA-5 alpha chain | ITGA5 | BD |
| | | | Pharmingen |
| CD49f | Integrin a6 chain, VLA-6 alpha chain, platelet gpIc | ITGA6 | BD |
| | | | Pharmingen |
| CD51 | Integrin alpha chain, vitronectin receptor a chain | ITGAV | BD |
| | | | Pharmingen |
| CD54 | ICAM-1, intercellular adhesion molecule 1 | ICAM1 | BD |
| | | | Pharmingen |
| CD55 | Decay-accelerating factor (DAF, prevent cell | DAF | BD |
| | damage) | | PharMingen |
| CD56 | Neural cell adhesion molecule 1 (NCAM1), NKH-1 | NCAM1 | Caltag |
| | (NK cell marker) | | |
| CD58 | Lymphocyte function-associated antigen-3 (LFA-3) | CD58 | BD |
| | | | PharMingen |
| CD59 | MACIF, MIRL, P-18, protectin | CD59 | BD |
| | | | Pharmingen |
| CD61 | Glycoprotein IIIa, beta3 integrin | ITGB3 | BD |
| | | | Pharmingen |
| CD62E | E-selectin, LECAM-2, ELAM-1 | SELE | BD |
| | | | Pharmingen |
| CD62L | L-selectin, LAM-1, Mel-14 | SELL | BD |
| | | | Pharmingen |
| CD62P | P-selectin, granule membrane protein-140 (GMP- | SELP | BD |
| | 140) | | Pharmingen |
| CD63 | LIMP, gp55, LAMP-3 neuroglandular antigen, | CD63 | BD |
| | granulophysin | | Pharmingen |
| CD64 | FcgR1, FcgammaR1 (IgG-receptor) | FCGR1A | Caltag |
| CD69 | Early T-cell activation antigen | CD69 | BD |
| | | | PharMingen |
| CD71 | Transferrin receptor | TFRC | BD |
| | | | Pharmingen |
| CD79a | CD79A antigen (immunoglobulin-associated alpha), | CD79A | BD |
| | MB1 | | PharMingen |
| CD79b | CD79B antigen (immunoglobulin-associated beta), | CD79B | BD |
| | B29 | | PharMingen |

TABLE 4-B
CELL SURFACE MARKERS USED FOR IMMUNOPHENOTYPING

| | | Gene | |
|---|---|---|---|
| | MOLECULE | Name | Source |
| CD80 | B7-1; BB1 | CD80 | BD |
| | | | Pharmingen |
| CD81 | Target of an antiproliferative antibody (TAPA-1); | CD81 | BD |
| | M38 | | Pharmingen |
| CD86 | B7-2; B70 | CD86 | Caltag |
| CD87 | Plasminogen activator, urokinase receptor | PLAUR | BD |
| | (PLAUR), uPAR | | PharMingen |
| CD90 | Thy-1 | THY1 | BD |
| | | | PharMingen |
| CD95 | APO-1, Fas, TNFRSF6 | TNFRSF6 | BD |
| | | | Pharmingen |
| CD100 | SEMA4D | SEMA4D | Serotec |
| CD104 | Integrin beta 4 subunit, TSP-1180 | ITGB4 | BD |
| | | | Pharmingen |
| CD105 | Endoglin | ENG | Caltag |
| CD106 | VCAM-1 (vascular cell adhesion molecule-1), | VCAM1 | BD |
| | INCAM-110 | | Pharmingen |
| CD117 | SCFR, c-kit, stem cell factor receptor | KIT | BD |
| | | | Biosciences |
| CD119 | IFN gamma receptor alpha chain (GIR-208) | IFNGR1 | BD |
| | | | Pharmingen |
| CD120a | TNFRI; TNFRp55 | TNFRSF1A | Serotec |
| CD126 | IL-6 receptor alpha chain | IL6R | BD |
| | | | Pharmingen |
| CD134 | Tumor Necrosis Factor receptor 4, OX40 | TNFRSF4 | Caltag |
| | (Promotes expression of BCL2/BCL-xL) | | |
| CD135 | fms-related tyrosine kinase 3 | FLT3 | BD |
| | | | PharMingen |
| CD140a | a-platelet derived growth factor (PDGF) receptor | PDGFRA | BD |
| | | | Pharmingen |
| CD140b | b-platelet derived growth factor (PDGF) receptor | PDGFRB | BD |
| | | | Pharmingen |
| CD141 | Thrombomodulin (TM), fetomodulin | THBD | BD |
| | | | Pharmingen |
| CD142 | Tissue factor, thromboplastin, coagulation factor III | F3 | BD |
| | | | Pharmingen |
| CD146 | Muc 18, MCAM, Mel-CAM, s-endo | MCAM | BD |
| | | | Pharmingen |
| CD147 | Basigin, M6, extracellular metalloproteinase | BSG | Serotec |
| | inducer (EMMPRIN) | | |
| CD152 | Cytolytic T lymphocyte-associated antigen (CTLA- | CTLA4 | BD |
| | 4) | | PharMingen |
| CD163 | M130, GHI/61, RM3/1 | CD163 | BD |
| | | | PharMingen |
| CD178 | CD95L, Fas Ligand | FASLG | Caltag |
| CD181 | CXCR1 CDw128a: IL-8 receptor alpha, | IL8RA | BD |
| | | | Pharmingen |
| CD182 | CXCR2 | IL8RB | BD |
| | | | Pharmingen |
| CD183 | CXCR3 chemokine receptor, G protein-coupled | CXCR3 | BD |
| | receptor 9 | | Pharmingen |
| CD184 | CXCR4 chemokine receptor, Fusin | CXCR4 | BD |
| | | | Pharmingen |
| CD200 | OX2 (regulates Mf activity) | CD200 | BD |
| | | | PharMingen |
| CD273 | B7DC, PDL2 | PDCD1LG2 | BD |
| | | | Pharmingen |
| CD274 | B7H1, PDL1 | PDCD1LG1 | BD |
| | | | Pharmingen |
| b2- | Associates with HLA class I antigen complex | B2M | BD |
| microglobulin | | | Pharmingen |
| EGFr | Epidermal growth factor receptor (growth and | EGFR | BD |
| | differentiation) | | PharMingen |
| HLA-ABC | MHC Class I ABC | | BD |
| | | | Pharmingen |
| HLA-DP | MHC II HLA-DP beta-1 | HLA- | Serotec |
| | | DPB1 | |
| NGFr | CD271, TNFR superfamily member 16 | NGFR | BD |
| | | | Pharmingen |
| RANTES | CCL5 (chemokine (C-C motif) ligand 5 | CCL5 | BD |
| | | | Pharmingen |

TABLE 5
AN EXAMPLE OF A PHENOTYPIC MARKER ARRAY PLATE FOR IMMUNOPHENOTYPING PBMC'S

| | 1 | 2 | 3 | 4 | 5 | 6 | 7 | 8 | 9 | 10 | 11 | 12 |
|---|---|---|---|---|---|---|---|---|---|---|---|---|
| A | Unstained | Unstained | Unstained | Unstained | Unstained | | | | | | | |
| B | Unstained | CD3 | CD4 | CD8 | CD11a | CD25 | CD28 | CD29 | CD35 | CD49c | CD56 | CD63 |
| C | CD71 | CD200 | b2- | CD64 | CD128a | | | | | | | |
| | | | microglobulin | | | | | | | | | |
| D | | | | | | | | | | | | |
| E | | | | | | | | | | | | |
| F | | | | | | | | | | | | |
| G | | | | | | | | | | | | |
| H | | | | | | | | | | | | |

TABLE 6
AN EXAMPLE OF A PHENOTYPIC MARKER ARRAY PLATE FOR IMMUNOPHENOTYPING PBMC'S

| | 1 | 2 | 3 | 4 | 5 | 6 | 7 | 8 | 9 | 10 | 11 | 12 |
|---|---|---|---|---|---|---|---|---|---|---|---|---|
| A | Unstained | Unstained | Unstained | Unstained | Unstained | Unstained | CD3 | CD4 | CD5 | CD7 | CD8 | CD9 |
| B | CD10 | CD11a | CD13 | CD14 | CD15 | CD16 | CD18 | CD19 | CD20 | CD22 | CD25 | CD29 |
| C | CD30 | CD31 | CD32 | CD33 | CD34 | CD35 | CD36 | CD38 | CD40 | CD44 | CD45 | CD49a |
| D | CD49b | CD49c | CD49d | CD49e | CD49f | CD51 | CD54 | CD55 | CD56 | CD58 | CD59 | CD61 |
| E | CD62E | CD62L | CD62P | CD63 | CD64 | CD69 | CD71 | CD73 | CD79a | CD79b | CD80 | CD81 |
| F | CD86 | CD87 | CD90 | CD95 | CD100 | CD103 | CD104 | CD105 | CD106 | CD117 | CD119 | CD120a |
| G | CD126 | CD134 | CD135 | CD140a | CD140b | CD141 | CD142 | CD146 | CD147 | CD152 | CD163 | CD178 |
| | | | | | | | | | | | | (95L) |
| H | CD181 (128a) | CD182 | CD183 | CD184 | CD200 | CD273 | CD274 | B2- | EGFR | HLA- | HLA-1 | RANTES |
| | | (128b) | | | | | | micorglobulin | | ABC | (DP) | |

TABLE 7
PHENOTYPIC DIFFERENCES BETWEEN THE CANCER CELL
LINES M21, DU-145, MCF-7, PC-3, BT-20, MDA-MB-231 AND HUVECS.

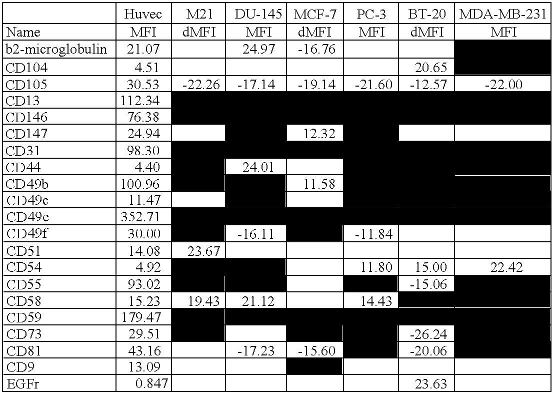

Read is upregulated, blue is downregulated. A dMFI of 25 is considered significant.

Red is upregulated, blue is downregulated. A dMFI of 25 is considered significant.

TABLE 8
PHENOTYPIC DIFFERENCES BETWEEN BREAST CANCER CELL
LINES MCF-7, BT-20 AND MDA-MB-231.

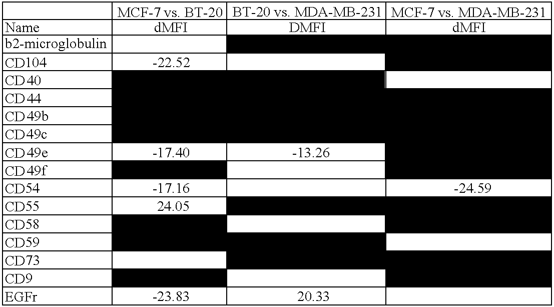

When comparing cell line a vs. b, dMFI calculated by subracting b from a.
Red: a is upregulated compared to b.
Blue: a is downregulated compared to b.

When comparing cell line a vs. b, dMFI calculated by subtracting b from a. Red: a is upregulated compared to b. Blue: a is downregulated compared to b.

TABLE 9
PHENOTYPIC DIFFERENCES BETWEEN PROSTATE CANCER
CELL LINES DU-145 AND PC-3

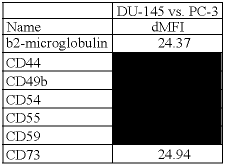

When comparing cell line a vs. b, dMFI calculated by subtracting b from a.
Red: a is upregulated compared to b.
Blue: a is downregulated compared to b.

When comparing cell line a vs. b, dMFI calculated by subtracting b from a. Red: a is upregulated compared to b. Blue: a is downregulated compared to b.

TABLE 10
PHENOTYPIC DIFFERENCES BETWEEN BREAST CANCER
CELL LINES MCF-7, BT-20 AND MDA-MB-231,
PROSTATE CANCER CELL LINES
DU-145 AND PC-3 AND M21 MELANOMA CELLS.

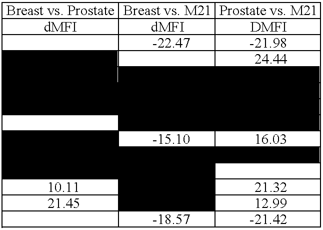

When comparing cell line a vs. b, dMFI calculated by subtracting b from a.
Red: a is upregulated compared to b.
Blue: a is downregulated compared to b.

When comparing cell line a vs. b, dMFI calculated by subtracting b from a. Red: a is upregulated compared to b. Blue: a is downregulated compared to b.

TABLE 11
ANALYSIS OF SURFACE MARKERS OF CANCER CELL LINES
GROUPED BY RESPONSIVENESS TO AN ANTI-TUMOR DRUG.

| | Low | Intermediate | High |
|---|---|---|---|
| | responsiveness | responsiveness | responsiveness |
| CD104 | − | − | + |
| CD146 | + | − | − |
| CD58 | +/− | +/− | ++ |

−: MFI < 10,
+/−: MFI 10-25,
+: MFI 25-75,
++: MFI > 75.

TABLE 12
MARKERS THAT SIGNIFICANTLY DIFFER AMONG CANCER
CELL LINES AND HUVECS.

| Name | Molecule | Function | Example of relation to cancer |
|---|---|---|---|
| CD9 | Type III | Signal transduction | Inversely associated with |
| | transmembrane | | metastasis⁹ |
| | protein | | |
| CD13 | Amino peptidase N | Unclear in epithelial cells | CD13 Gene defects cause |
| | | | leukemia and lymphoma |
| CD31 | PECAM1 | Adhesion molecule | Inversely associated with breast |
| | | | cancer morphology and |
| | | | invasiveness¹⁰ |
| CD40 | TNF receptor | Signal transduction | CD40 has pro- and anti-apoptotic |
| | superfamily 5 | | properties in cance¹¹ |
| CD44 | Pgp1 | Cell-cell interactions, adhesion | Associated with cancer |
| | | and migration | metastasis¹² |
| CD49b | Integrin α2 | Adhesion and cell-signaling | Inversely related to |
| | | | immortalization and tumor |
| | | | progression¹³ |
| CD49c | Integrin α3 | Cell adhesion and cell-matrix | Tumor invasiveness¹⁴ |
| | | interactions | |
| CD49e | Integrin α5 | Adhesion and cell-signaling | Tumor suppressor activity¹⁵ |
| CD49f | Integrin α6 | Adhesion and cell-signaling | Activated during carcinoma |
| | | | progression¹⁶ |
| CD51 | Integrin alpha V | Adhesion and signal | Metastasis of melanoma cells¹⁷ |
| | receptor | transduction | |
| CD54 | ICAM-1 | Adhesion molecule | Expressed in metastatic |
| | | | melanoma¹⁸ |
| CD55 | DAF | Regulates complement attack | Increased expression attenuates |
| | | | prostate cancer¹⁹ |
| CD58 | LFA-3 | Adhesion molecule | Binds p53 tumor suppressor |
| | | | protein²⁰ |
| CD59 | Complement | Regulates complement attack | Increased expression protects |
| | regulatory protein | | cancer cells from complement |
| | | | attack²¹ |
| CD73 | Ecto′5 | Cell signaling. Production of | Adenosine is important in tumor |
| | nucleotidase | adenosine. | growth and progression²² |
| CD81 | Type IV | Signal transduction. | Localized in tumor suppressor |
| | transmembrane | Development, growth and | gene region |
| | protein | motility | |
| CD104 | Integrin β4 | Cell adhesion | α6β4-integrin contributes to |
| | | | carcinoma progression¹⁶ |
| CD105 | Endoglin | Component of TGF-beta | Modulates proliferative rate of |
| | | receptor complex | solid tumor cells²³ |
| CD146 | MCAM | Adhesion molecule | Increased expression in metastatic |
| | | | melanoma cell lines²⁴ |
| CD147 | Basigin | Intercellular recognition | Mediator of malignant cell |
| | | | behavior²⁵ |
| b2- | b2-microglobulin | Mediates MHC class I | MHC Class I mediates T cell and |
| microglobulin | | expression | NK cell recognition of cancer cells |
| EGFR | EGFR | Cell signal, cell growth | Overexpression correlates with |
| | | | depth of tumor invasion²⁶ |

Publications cited throughout this document are hereby incorporated by reference in their entirety. Although the various aspects of the invention have been illustrated above by reference to examples and embodiments, it will be appreciated that the scope of the invention is defined not by the foregoing description, but by the following claims properly construed under principles of patent law.

What is claimed is:

1) A system for comparing the phenotypic state of a first unknown sample material with a sample material of a known phenotypic state.

2) The system of claim 1 comprising:
a. A panel of phenotypic markers, and;
b. Reagents to detect the presence of phenotypic markers in the first sample material of unknown phenotypic state, and;
c. Apparatus to record the presence or absence of phenotypic markers in the first sample material of unknown phenotypic state, and;
d. Reagents to detect the presence of phenotypic markers in the sample material of a known phenotypic state, and;
e. Apparatus to record the presence or absence of phenotypic markers in the sample material of a known phenotypic state, and;
f. Apparatus to report differences between the first unknown sample material and the sample material of a known phenotypic state.

3) The system of claim 1, wherein the panel of phenotypic markers is in a multiwell plate.

4) The system of claim 1, wherein the apparatus to record the presence of phenotypic markers is selected from the group consisting of a microscope, a flow cytometer, a plate reader, and a gel electrophoresis apparatus.

5) A device for comparing the phenotypic state of a first unknown sample material with a sample material of a known phenotypic state.

6) The device of claim 5 comprising:
a. A panel of phenotypic markers, and;
b. Reagents to detect the presence of phenotypic markers in the first sample material of unknown phenotypic state, and;
c. Apparatus to record the presence or absence of phenotypic markers in the first sample material of unknown phenotypic state, and;
d. Reagents to detect the presence of phenotypic markers in the sample material of a known phenotypic state, and;
e. Apparatus to record the presence or absence of phenotypic markers in the sample material of a known phenotypic state, and;
f. Apparatus to report differences between the first unknown sample material and the sample material of a known phenotypic state.

7) The device of claim 5, wherein the panel of phenotypic markers is in a multiwell plate.

8) The device of claim 5, wherein the apparatus to record the presence of phenotypic markers is selected from the group consisting of a microscope, a flow cytometer, a plate reader, and a gel electrophoresis apparatus.

9) A kit for comparing the phenotypic state of a first unknown sample material with a sample material of a known phenotypic state.

10) The kit of claim 9 comprising:
a. A panel of phenotypic markers, and;
b. Reagents to detect the presence of phenotypic markers in a sample material.

11) The kit of claim 10, wherein the reagents are in a multiwell plate.

12) A method for using the system of claim 1, comprising the steps of:
a. Obtaining a first unknown sample material, and;
b. Contacting the first unknown sample material with reagents to detect the presence of phenotypic markers in the first unknown sample material, and;
c. Recording the presence of phenotypic markers in the first unknown sample material, and;
d. Obtaining a sample material of a known phenotypic state, and;
e. Contacting the sample material of a known phenotypic state with reagents to detect the presence of phenotypic markers in the sample material of a known phenotypic state, and;
f. Recording reagents the presence of phenotypic markers in the sample material of a known phenotypic state, and;
g. Reporting the differences between the unknown sample material and the sample material of a known phenotypic state.

13) A method for using the kit of claim 9, comprising the steps of:
a. Obtaining a first unknown sample material, and;
b. Contacting the first unknown sample material with a kit containing reagents to detect the presence of phenotypic markers in the first unknown sample material, and;
c. Recording the presence of phenotypic markers in the first unknown sample material, and;
d. Obtaining a sample material of a known phenotypic state, and;
e. Contacting the sample material of a known phenotypic state with a kit containing reagents to detect the presence of phenotypic markers in the sample material of a known phenotypic state, and;
f. Recording reagents the presence of phenotypic markers in the sample material of a known phenotypic state, and;
g. Reporting the differences between the unknown sample material and the sample material of a known phenotypic state.
